(19) United States
(12) Patent Application Publication
(10) Pub. No.: US 2013/0052709 A1
(43) Pub. Date: Feb. 28, 2013

(54) SYSTEMS AND PROCESSES FOR PRODUCING BIOFUELS FROM BIOMASS

(71) Applicants: Gary Wietgrefe, Sioux Falls, SD (US); Thomas Bregger, wake Forest, NC (US)

(72) Inventors: Gary Wietgrefe, Sioux Falls, SD (US); Thomas Bregger, wake Forest, NC (US)

(73) Assignee: SYNGENTA PARTICIPATIONS AG, Basel (CH)

(21) Appl. No.: 13/575,640

(22) PCT Filed: Feb. 9, 2011
(86) PCT No.: PCT/US11/24124

§ 371 Date: Oct. 23, 2012

Related U.S. Application Data
(60) Provisional application No. 61/302,761, filed on Feb. 9, 2010.

(51) Int. Cl. C12P 7/10; C12P 7/14

(52) U.S. Cl. 435/162; 435/165

(57) ABSTRACT

Systems and processes for converting bulky lignocellulosic biomass to high density biomass products, including biofuels, are described. The systems and processes relate to treating freshly harvested plant materials, generally at or in close proximity to sites where the plant materials are harvested, to effect saccharification, alcoholic fermentation, or simultaneous saccharification and fermentation, thereby providing a biomass slurry. The biomass slurry is extracted to provide liquid extracts comprising biomass-derived water and water soluble biomass saccharification and fermentation products, including fermentable sugars and alcohols. The biomass slurry extracts can be transported via pipeline to other locations for fermentation, further saccharification, and/or purification to provide biofuel. Alternatively, the biomass slurry can be used to prepare a biomass slurry that can be transported via pipeline.

### CROSS-REFERENCE TO RELATED APPLICATIONS

This application claims priority to U.S. Provisional Application Ser. No. 61/302,761 filed on Feb. 9, 2010.

### TECHNICAL FIELD

The presently disclosed subject matter provides processes for converting lignocellulosic biomass to a biomass slurry and to biofuels, such as bioethanol, ethanol, ethanol/gasoline blends and other bioalcohols.

### BACKGROUND

Cellulosic and lignocellulosic feedstocks (e.g., plant-derived biomass) provide a large renewable source of potential starting materials for the production of a variety of chemicals, plastics, fuels and feeds. For example, biomass feedstocks comprise a variety of carbohydrates which can be hydrolyzed to provide fermentable sugars for use in the production of alcohol fuels, such as ethanol, methanol, and butanol.

The use of biomass feedstocks for production of biofuels is motivated by both economic and environmental concerns, including reduction of greenhouse gas emissions, enhancement of the fuel supply, and maintenance of the rural economy. Energy legislation enacted in 2007 in the United States provides that yearly ethanol production reach 136.3 billion liters by the year 2022, with at least 79.5 billion liters coming from lignocellulosic feedstocks, such as corn stover, prairie grass, and poplar trees, as opposed to corn grain, which comprises a high amount of more easily hydrolyzed starch. See, e.g., Ethanol Producer Magazine, December 2007.

There is a continuing need for improved processes and systems for converting biomass to biofuels and/or high density biofuel feedstocks. In particular, there is a need for efficient processes that can reduce biomass storage and transport issues, and which do not require the use of large amounts of externally provided water.

### SUMMARY

In some embodiments, the presently disclosed subject matter provides a process for converting plant biomass to a biomass slurry comprising ethanol; the process comprising: providing biomass, wherein providing the biomass comprises collecting a plant biomass, wherein the collecting is timed to provide a plant biomass comprising a moisture content of between about 70% and about 95%; chopping the plant biomass and placing the chopped biomass into a chamber for a period of time and under suitable conditions to effect saccharification of the biomass, alcohol fermentation of the biomass, or combinations thereof, thereby providing a biomass slurry, wherein the biomass slurry comprises residual solids, biomass-derived water, and water-soluble products, the water soluble products comprising fermentable sugars and/or an alcohol. Furthermore, the biomass slurry can be physically separated into a solid and liquid fraction where the liquid fraction is biomass slurry extract and the solid fraction is residual solids.

In some embodiments, the chopping comprises chopping the biomass to a theoretical length of cut (TLC) between about 0.3 and about 1.3 centimeters. In some embodiments it may be beneficial to chop the biomass material twice or more or to grind the biomass to a fine mash or powder.

In some embodiments, the suitable conditions further comprise providing an inoculant comprising one or more biomass-processing biocatalyst. In some embodiments, the inoculant is added to the biomass prior to or during placing the biomass into the chamber. In some embodiments, the biomass-processing biocatalyst comprises one or more of a lignocellulose-processing enzyme and an alcohol-producing microbe.

In some embodiments, the inoculant further comprises one or more additive selected from the group consisting of a biocatalyst nutrient, a biocatalyst growth factor, a pH-adjusting agent, an electrolyte, a nitrogen-containing chemical, an antimicrobial agent, an oxygen-depleting agent, a beneficial microbe, a plasticizer, a softener, and combinations thereof. In some embodiments, the oxygen-depleting agent is selected from the group consisting of CO₂ gas and chloropicrin. In some embodiments, the nitrogen-containing chemical is selected from the group consisting of ammonia, ammonium chloride, urea, ammonium nitrate, and ammonium phosphate.

In some embodiments, the process further comprises monitoring contents of the chamber at one or more locations in the chamber to determine one or more of the pH, temperature, oxygen gas content, escaping gases, microbial activity, enzymatic activity, percent dry matter (DM) conversion, percent of theoretical sugars converted, fermentable sugars concentration, alcohol concentration, plant biomass-derived acid concentration, and microbial nutrient concentration. In some embodiments, conditions in the chamber are adjusted during the period of time to alter one or more of pH, temperature, oxygen gas content, microbial activity, enzymatic activity, and microbial nutrient content.

In some embodiments, the plant biomass is derived from a plant selected from one or more of the group consisting of maize, soybean, millet, milo, rye, wheat, triticale, oats, barley, rice, sorghum, sudangrass, switchgrass, Miscanthus, alfalfa, cotton, sisal, hemp, jute, turf grass, rape, sunflower, willow, eucalyptus, poplar, pine, willow, tobacco, clover, bamboo, flax, pea, radish, turnip, potato, sweet potato, cassaya, taro, beet, sugar beet, sugar cane, and canola. In some embodiments, the biomass comprises one or more of the group consisting of whole plant corn, corn stover, corn cobs, and soybean forage. In some embodiments, the collecting is timed to provide a plant biomass comprising a moisture content of about 75% or more.

In some embodiments, the biomass comprises plant material selected based on one or more characteristic of the group consisting of sugar content, cellulose content, lignin content, cost, growing season, drought resistance, disease resistance, individual plant size, and tonnage. In some embodiments, the biomass comprises plant material from a male-sterile, tropical hybrid corn plant.

In some embodiments, at least a portion of the plant biomass is derived from a transgenic plant. In some embodiments, the transgenic plant comprises one or more lignocellulose-processing enzyme. In some embodiments, the lignocellulose-processing enzyme is an amylase. Examples of transgenic plants comprising one or more lignocellulose-processing enzymes and methods of use may be found in U.S. Patent Publication 2003-0135885 A1 herein incorporated by reference.

In some embodiments, the chamber is an upright silo.

In some embodiments, the period of time is from about 20 hours to about 21 days. In some embodiments, the period of time is from about 24 hours to about 72 hours. In some embodiments the time period from collecting of plant biomass to generation of biomass slurry is 12 hours or less.

In some embodiments, the physically separating comprises one or more of centrifuging, pressing, and decanting. In some embodiments, the biomass slurry extract comprises water soluble products and at least about 80% of the biomass-derived water, and the residual solids comprises about 20% of the biomass-derived water.

In some embodiments, the plant biomass is collected at a first location and the chamber is at a location at or in close proximity to the first location. In some embodiments, the process further comprises transporting the biomass slurry or the biomass slurry extract to a second location; and treating the biomass slurry or biomass slurry extract to provide a biofuel. In some embodiments, the second location is an ethanol plant. In some embodiments, one or more of pH, enzymatic activity level, microbial activity level, and viscosity of the biomass slurry or biomass slurry extract is adjusted prior to the transporting.

In some embodiments, treating the biomass slurry comprises saccharifying water soluble carbohydrates in the biomass slurry. In some embodiments, purifying the biomass slurry to provide a purified alcohol comprises separating alcohol from biomass-derived water by one or more of distilling and drying over molecular sieves. In some embodiments, the purified alcohol is mixed with gasoline to provide the biofuel.

In some embodiments, the process further comprises treating the residual solids fraction to provide one or more co-products selected from the group consisting of an animal feed, a fertilizer, methanol, and a boiler fuel. In some embodiments, the process further comprises collecting the biomass-derived water from the biomass slurry extract and using the biomass-derived water for one or more of irrigating a biomass plant material prior to collecting, diluting the biomass or biomass slurry extract for saccharification, fermentation, or saccharification and fermentation, processing of the residual solids fraction, and distilling a biofuel.

In some embodiments, providing biomass further comprises freezing the biomass to control endogenous microbes, to break down lignocellulosic materials within the biomass, or combinations thereof.

In some embodiments, conditions within the chamber comprise heat generated by one or more of plant cell respiration, microbial activity, and enzymatic activity. In some embodiments, the heat generated by one or more of plant cell respiration, microbial activity, and enzymatic activity is controlled by one or more of biomass moisture, ambient harvest temperature, biomass theoretical length of cut (TLC), oxygen content of the chamber, nutrients, pH, inoculant load, inoculant type, and a heat exchange system. In some embodiments, the heat generated by one or more of plant cell respiration, microbial activity, and enzymatic activity provides the biomass slurry in the absence of additional heat.

An object of the presently disclosed subject matter having been stated hereinabove, and which is achieved in whole or in part by the presently disclosed subject matter, other objects will become evident as the description proceeds when taken in connection with the accompanying drawings as best described herein below.

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist.

### I. DEFINITIONS

Following long-standing patent law convention, the terms “a” and “an” mean “one or more” when used in this application, including the claims. Thus, “an enzyme” or “a plant material” can refer to a plurality (i.e., two or more) of enzymes or plant materials.

As used herein, the term “about” modifying any amount can refer to the variation in that amount encountered in real world conditions of producing sugars and ethanol, e.g., in the lab, pilot plant, or production facility. For example, the amounts can vary by about 5%, 1%, or 0.5%. Unless otherwise indicated, all numbers expressing quantities of percentage (%), temperature, time, pH, distance, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

The term “saccharide” refers to a carbohydrate monomer, oligomer or larger polymer. Thus, a saccharide can be a compound that includes one or more cyclized monomer unit based upon an open chain form of a compound having the chemical structure H(CHOH)ₙC(═O)(CHOH)ₘH, wherein the sum of n+m is an integer between 2 and 8. Thus, the monomer units can include trioses, tetroses, pentoses, hexoses, heptoses, nonoses, and mixtures thereof. In some embodiments, each cyclized monomer unit is based on a compound having a chemical structure wherein n+m is 4 or 5. Thus, saccharides can include monosaccharides including, but not limited to, aldohexoses, aldopentoses, ketohexoses, and ketopentoses such as arabinose, lyxose, ribose, xylose, ribulose, xylulose, allose, altrose, galactose, glucose, gulose, idose, mannose, talose, fructose, psicose, sorbose, and tagatose, and to hetero- and homopolymers thereof. Saccharides can also include disaccharides including, but not limited to sucrose, maltose, lactose, trehalose, and cellobiose, as well as hetero- and homopolymers thereof.

The term “oligosaccharide” refers to polysaccharides having a degree of polymerization of between about 2 and about 10.

The terms “fermentable sugar” and “sugar” can be used interchangeably and refer to oligosaccharides, monosaccharides and mixtures thereof that can be used as a carbon source in a fermentation process. Fermentable monosaccharides include arabinose, glyceraldehyde, dihydroxyacetone, erythrose, ribose, ribulose, xylose, glucose, galactose, mannose, fucose, fructose, sedoheptulose, neuraminic acid, or mixtures of these. Fermentable disaccharides include sucrose, lactose, maltose, gentiobiose, or mixtures thereof.

As used herein the term “starch” refers to a polysaccharide polymer of glucose containing α(1-4) and α(1-6) glycosidic bonds. In particular, starch refers to a mixture of amylose and amylopectin.

The term “dextrin” refers to a linear, water-soluble oligomer of α-(1-4)-D-glucose. Dextrins can be prepared from the hydrolysis of starch.

The term “cellulose” refers to a polysaccharide of β-glucose comprising β-(1-4) glycosidic bonds. The term “cellulosic” refers to a composition comprising cellulose.

The terms “glycosidic bond” and “glycosidic linkage” refer to a linkage between the hemiacetal group of a saccharide and the hydroxyl group of an alcohol (which can be another saccharide).

The term “lignocellulosic” refers to a composition comprising both lignin and cellulose. In some embodiments, lignocellulosic material can comprise hemicellulose, a polysaccharide which can comprise saccharide monomers other than glucose. In some embodiments, the lignocellulosic material can also comprise starch.

“Lignin” is a polyphenolic material. Lignins can be highly branched and can also be crosslinked. Lignins can have significant structural variation that depends, at least in part, on the plant source involved.

Lignocellulosic materials include a variety of plants and plant materials, such as, but not limited to, papermaking sludge; wood, and wood-related materials, e.g., saw dust, or particle board, leaves, or trees, such as poplar trees; grasses, such as switchgrass; whole plant corn; sorghums; sudangrass; grass clippings; rice hulls; bagasse (e.g., sugar cane bagasse); jute; hemp; flax; bamboo; sisal; abaca; hays; straws; corn cobs; corn and sorghum stover; and coconut hair.

The term “biofuel” refers to a fuel that is derived from biomass, i.e., a living or recently living biological organism, such as a plant or an animal waste. Biofuels include, but are not limited to, biodiesel, biohydrogen, biogas, biomass-derived dimethylfuran (DMF), and the like. In particular, the term “biofuel” can be used to refer to biomass-derived alcohols (e.g., bioalcohol), such as ethanol, methanol, propanol, or butanol, which can be denatured, if desired prior to use. The term “biofuel” can also be used to refer to fuel mixtures comprising biomass-derived fuels, such as alcohol/gasoline mixtures (i.e., gasohols). Gasohols can comprise any desired percentage of biomass-derived alcohol (i.e., about 10%, 15%, 20%, 25%, 30%, 35%, 40%, 45%, 50%, 55%, 60%, 65%, 70%, 75%, 80%, 85%, 90%, or 95% biomass-derived alcohol). For example, one useful biofuel-based mixture is E85, which comprises 85% ethanol and 15% gasoline.

The term “biocatalyst” refers to both enzymatic catalysts and microbes (e.g., bacteria, fungi, etc.) that produce enzymatic catalysts or otherwise act as catalysts. Biocatalysts can catalyze (e.g., increase the rate of or otherwise facilitate) conversion of one molecule to another. Thus, biocatalysts can catalyze a variety of chemical reactions, such as hydrolysis reactions, isomerization reactions, and the like.

The term “enzyme” refers to a protein that catalyzes the conversion of one molecule into another. The term “enzyme” as used herein includes any enzyme that can catalyze the transformation of a biomass-derived molecule to another biomass-derived molecule. In particular, enzymes include those which can degrade polysaccharides (e.g., cellulose, starch, hemicellulose, or lignocellulose molecules) to provide fermentable sugars and alcohols. Enzymes also include those which can convert one type of sugar into another type of sugar. Enzymes that degrade polysaccharides or that can transform one type of sugar into another can also be referred to herein as “lignocellulose-processing enzymes” or “biomass-processing enzymes”.

The term “ethanol-producing biocatalyst microbe” refers to any microbe that can convert a carbohydrate or sugar into ethanol. Suitable ethanol-producing biocatalyst microbes can be micro-organisms selected from bacteria, filamentous fungi, and yeast. The ethanol-producing biocatalyst microbe can be a wild type micro-organism, a mutated micro-organism, or a recombinant micro-organism and can include, for example, Escherichia, Zymomonas, Saccharomyces, Candida, Pichia, Streptomyces, Bacillus, Lactobacillus, and Clostridium. In some embodiments, the ethanol-producing biocatalyst microbe can be selected from the group consisting of recombinant Escherichia coli, Zymomonas mobilis, Bacillus stearothermophilus, Saccharomyces cerevisiae, Clostridia thermocellum, Thermoanaerobacterium saccharolyticum, and Pichia stipitis. In some embodiments, the fermentation or other biomass-processing microbe of the presently disclosed processes is an anaerobic microbe.

For use in a process of the presently disclosed subject matter, an enzyme can be specifically selected based on the specific end product desired from the biomass. The enzyme can also be selected to provide a desired property for the biomass contained in a chamber or to the liquid obtained from the contained biomass. For example, an enzyme can be selected in order to produce a biomass product of desired viscosity or pH.

As used herein the terms “liquefaction,” “liquefy,” “liquefact,” “liquefied” and variations thereof refer to the process or the products of processes related to increasing the amount of water soluble molecules (e.g., water soluble carbohydrates) in plant-derived biomass. In some embodiments, the term “liquefy” can apply to fermenting (e.g. fermenting to provide an alcohol), saccharifying, or combinations thereof.

As used herein, the terms “hydrolyze,” “saccharification,” “saccharifying,” and variations thereof refer to the process of converting polysaccharides (e.g., cellulose or starch) to fermentable sugars, e.g., through the hydrolysis of glycosidic bonds. Saccharification can be effected with enzymes. The enzymes can be produced in the plant or added to biomass directly (e.g., as a solid or liquid enzyme additive) or can be produced in situ by microbes (e.g., yeasts, fungi, bacteria, etc.). Saccharification products include, for example, fermentable sugars, such as glucose and other small (low molecular weight) oligosaccharides such as monosaccharides, disaccharides, and trisaccharides. Saccharification products can also simply include lower molecular weight polysaccharides than those in the original cellulose or lignocellulose. “Suitable conditions” for saccharification refer to various conditions including pH, temperature, moisture, nutrients, biomass composition, and inoculant composition.

“Fermentation” or “fermenting” can refer to the process of transforming an organic molecule into another molecule using a micro-organism. For example, “fermentation” can refer to transforming sugars or other molecules from biomass to produce alcohols (e.g., ethanol, methanol, butanol); organic acids (e.g., citric acid, acetic acid, itaconic acid, lactic acid, gluconic acid); ketones (e.g., acetone), amino acids (e.g., glutamic acid); gases (e.g., H₂ and CO₂), antibiotics (e.g., penicillin and tetracycline); enzymes; vitamins (e.g., riboflavin, B₁₂, beta-carotene); and/or hormones. Thus, fermentation includes alcohol fermentation. Fermentation also includes anaerobic fermentations.

Fermenting can be accomplished by any organism suitable for use in a desired fermentation step, including, but not limited to, bacteria, fungi, archaea, and protists. Suitable fermenting organisms include those that can convert mono-, di-, and trisaccharides, especially glucose and maltose, or any other biomass-derived molecule, directly or indirectly to the desired fermentation product (e.g., ethanol, butanol, etc.). Suitable fermenting organisms also include those which can convert non-sugar molecules to desired fermentation products.

In some embodiments, the fermenting is effected by a fungal organism (e.g., yeast or filamentous fungi). The yeast can include strains from a Pichia or Saccharomyces species. In some embodiments, the yeast can be Saccharomyces cerevisiae. In some embodiments, the fermenting is effected by bacteria. For example, the bacteria can be Clostridium acetobutylicum (e.g., when butanol is the desired fermentation product) or Corynebacterium glutamicum (e.g., when monosodium glutamate (MSG) is the desired fermentation product). In some embodiments, the micro-organism (e.g. yeast or bacteria) can be a genetically modified micro-organism. In some instances, the organism can be yeast or other organism having or modified to be active in the presence of high concentrations of alcohol.

The term “alcohol fermentation” refers to the conversion of a fermentable sugar to an alcohol (e.g., methanol, ethanol, propanol, butanol, etc.). The particular product of a given alcohol fermentation can be determined by the biocatalyst used in the fermentation and/or the substrate of fermentation (i.e., the type of fermentable sugar being converted).

In certain embodiments, fermenting can comprise contacting a mixture including biomass-derived sugars with an alcohol-producing biocatalyst, such as yeast or another alcohol-producing microbe. In some embodiments, fermenting involves simultaneous saccharification and fermentation (SSF). The amount of fermentation biocatalyst employed can be selected to effectively produce a desired amount of ethanol in a suitable time and/or upon the sugar content of a given fermentation mixture. The use of alcohol-producing biocatalyst can increase the rate of saccharification by reducing the concentration of sugars, which can inhibit saccharification biocatalysts.

“Suitable conditions” for alcohol fermentation can refer to conditions that support the production of ethanol or another alcohol by a biocatalyst. Such conditions can include pH, nutrients, temperature, moisture, atmosphere, and other factors.

The term “inoculant” refers to any chemical compound, biomolecule (e.g., enzyme) or organism, or mixtures thereof, which are added to freshly cut biomass or to biomass that is going into the chamber. Thus, an inoculant can include biomass-processing biocatalysts, including lignocellulose-processing enzymes and microbes (e.g., saccharifying enzymes saccharifying microbes, alcohol-producing enzymes, alcohol-producing microbes, etc.), sterilizing agents (e.g., anti-microbial or bactericidal agents), pH-adjusting agents, electrolytes, oxygen-depleting agents, nitrogen-containing agents, enzyme nutrients and cofactors, softening agents, plasticizers, fillers, and combinations thereof. A “microbe inoculants” refers to a subset of the term inoculants as defined comprising microbes which are added to freshly cut biomass or to biomass that is going into the chamber.

The term “pH-adjusting chemical” or “pH-adjusting agent” can refer to any chemical or agent added to control pH in the chamber containing biomass, extracted liquids, or solids. Thus, pH-adjusting chemicals and agents can include pH-lowering agents, including mineral acids, organic acids, and acid salts; pH-raising agents, such as bases (e.g., ammonia, and ammonium salts); and buffering agents. Suitable pH-adjusting acids include, but are not limited to, hydrochloric acid, sulfuric acid, citric acid, formic acid, propionic acid, acetic acid, lactic acid, butyric acid, phosphoric acid, and the like. Suitable pH-adjusting agents also include acid salts, such as sodium diacetate. Suitable bases include, but are not limited to, sodium hydroxide, Na₂CO₃, and ammonium hydroxide. Buffering agents include, but are not limited to, CaCO₃, NaHCO₃, NH₄Cl, NaH₂PO₄, K₂HPO₄, and KH₂PO₄. In some embodiments, the buffering agents can act as weak acids or bases, or simply to help maintain the pH within a desired range.

The term “oxygen-depleting agent” refers to additives that can be used in the chamber to hasten or maintain the achievement of an anaerobic environment in the chamber. Suitable oxygen-depleting agents include, but are not limited to, chloropicrin. Oxygen-depleting agents also include gases that can be used to displace oxygen, including CO₂ gas.

The terms “plasticizer” and “softening agent” refer to materials that cause a reduction in cohesive intermolecular forces along or between polymer chains. Such material can act, for example, to decrease crystallinity, or disrupt bonds between lignin and non-lignin carbohydrate fibers (e.g., cellulose or hemicellulose). Plasticizers and softening agents include, but are not limited to polyols (e.g., glycerol, ethylene glycol), esters of polyols (e.g., glycerol monoacetate), glycol ethers (e.g., diethylene glycol), acetamide, and ethanolamines.

The term “chamber” refers to a container inside which, fermentation reactions can be carried out and at least a portion of the total contents being fermented is maintained at anaerobic conditions. In some embodiments, the chamber is an airtight or air-limiting container. In some embodiments, the chamber is a glass-lined silo, such as commercially available silo originally designed for the ensiling of plant materials to provide animal fodder.

The phrase “at or in close proximity to” can be used to refer to actions or steps in a process that occur or are performed at about the same location or that would not involve transport or travel of appreciable distances. Thus, “at or close proximity to” can refer to actions or steps that occur or are performed within less than about a 8 kilometers radius (e.g., within less than about a 8, 6, 4, 3, 2, or 1 kilometer radius) of one another. In some embodiments, the steps that are performed at or in close proximity to one another are performed in less than about one kilometer of one another. Generally, actions taking place at or in close proximity to one another can occur on the same property, (e.g., on the same farm).

As used herein the terms “remote” and “remotely located” refer to a site or location from which trucking raw high density biomass is economically undesirable or otherwise inconvenient. Thus, a remotely located site refers to a site from which it is advantageous to transport high density biomass products via pipeline. In some embodiments, the remotely located site or location is at least about 8 kilometers from a delivery destination. In some embodiments, the remotely located site is at least about 16, 32, 48, 64, 80, 160, 400, or 800 kilometers from a delivery destination.

The term “transporting” can refer to moving a composition, such as a biomass slurry or biomass slurry extract, from one location to another (e.g., from a first location to a second location). Transporting can involve piping the composition via a pipeline or shipping the composition via ship, barge, tanker, truck, train, or airplane. In some embodiments, transporting comprises piping.

### II. CONVERSION OF BIOMASS TO BIOMASS SLURRY

Historically, the ensiling of surplus forage has proven a useful and convenient method to preserve feedstuffs for animals, particularly ruminant farm animals, such as cattle. Ensiling is a forage storage and preservation system. Ensiling usually involves primarily acid fermentation, wherein lactic acid bacteria present on the forage or added as an inoculant ferments water soluble carbohydrates to organic acids (e.g., lactic acid) under aerobic and, later, anaerobic conditions. The desired production of lactic acid causes a decrease in pH, which then inhibits any microbes present so that nutrients in the forage are preserved.

In particular, the presently disclosed process relates to a thermogenetic, enzymatic, chemical, and/or microbial conversion process to condense fresh plant biomass and, in some embodiments, to transport the biomass slurry comprising water soluble carbohydrates, fermentable sugars and/or biofuel-containing solutions to a fermentation or distillation facility. Thus, the presently disclosed subject matter generally relates to processes involving the solid-state saccharification and/or alcohol fermentation of biomass materials wherein the solid-state treatment comprises the primary or only saccharification step in the conversion of the raw plant biomass. The conditions can be controlled so that alcohol is the primary fermentation product or so that the sugars are extracted from the biomass being treated prior to significant (i.e., greater than 10%, 20%, 30%, 40% or 50%) conversion of the sugars to an acid fermentation product. In some embodiments, the treatment conditions are controlled to maximize alcoholic fermentation and/or such that the treatment comprises simultaneous saccharification and alcohol fermentation of the biomass.

Liquid from the biomass slurry is subsequently extracted to provide a biomass slurry extract comprising biomass-derived water and water-soluble biomass-derived molecules such as fermentable sugars and alcohols. The biomass slurry extract can also include water soluble polysaccharides that can be further saccharified and then fermented.

Treatment of the raw biomass materials can be accomplished using conventional harvesting and silo-related equipment, close to the site of biomass production. For example, plant biomass can be converted to biomass slurry on the farm on which the plant biomass is grown and harvested.

The heat needed to affect saccharification and/or alcohol fermentation of the biomass (i.e., to provide the biomass slurry) can be provided by the process itself through fresh plant cell respiration, enzymatic, and microbial activity. For example, the piling of the biomass in the chamber can raise the temperature of the biomass above the ambient temperature. Among other things, the density and moisture content of the piled biomass can affect the amount of temperature increase that can be achieved. In particular, the plant-derived heat can provide suitable conditions for the activity of endogenous and/or exogenous plant-degrading enzymes or microbes. By taking advantage of the heat produced by the enclosed pile of plant biomass and/or the enzymatic and microbial activity, the energy requirements typically needed for biomass saccharification can be considerably reduced. In particular, in some embodiments, provision of the biomass slurry (e.g., saccharification and, in some embodiments, partial alcohol fermentation) is accomplished in the absence of additional heat (i.e., in the absence of any externally provided heat, such as heat not produced by plant cell respiration, microbial/enzymatic activity, or the ambient temperature). “Externally provided heat” or “external heat” herein interchangeably refers to heat not produced in the chamber by plant cell respiration, microbial activity, enzymatic activity, or ambient temperature. For example, sources that may provide “external heat” may consists of but are not limited to solar power, gas heat, electric heating or hydrothermal heat.

The biomass density, and thus, the temperature within the chamber can be controlled by chopping the biomass. Herein, the terms “chop”, “chopping” or “chopped” may interchangeably refer to any means of homogenization or breaking down materials into smaller units including but not limited to, cutting, chopping, grinding, crushing, pressing, compaction and the like or combinations thereof. Commercially available silage choppers can be used to chop the biomass after harvesting. For example, the biomass can be chopped to a theoretical length of cut (TLC) of between about 0.3 and about 1.3 centimeters. The biomass can also be ground coarsely or to a fine mash or powder. It may also be chopped multiple times if necessary.

The size of the chamber, particularly the height of the chamber, can also affect the biomass density. In some embodiments, the chamber is an upright silo. Suitable silos include commercially available enameled steel silos, also known as glass-lined silos. In some embodiments the silos may be 25, 50, 75 to about 100 feet tall.

Generally, the biomass should not be allowed to dry prior to being introduced into the chamber (i.e., the plant material is “freshly harvested”). Thus, the biomass should be chopped and loaded into the chamber less than 24 hours after being harvested or collected. In some embodiments, the time from collection to chamber is even shorter (i.e., less than about 20, 18, 16, 14, 12, 10, 9, 8, 7, 6, 5, 4, 3, 2, or 1 hour). In some embodiments, the time from collection to chamber is within about 20 minutes. In some embodiments, the time from collection to chamber is within about 30, 40, 50, 60, 70, 80, 90, or 100 minutes. In some embodiments, maize, or other plants are harvested, chopped and placed into the chamber within a range of about 30 minutes to 100 minutes after collection. In some aspects of the invention, aerial or above ground maize, or other plants are collected, chopped and then placed in the chamber within a maximum of 30 minutes.

In order to aid in the saccharification and/or alcohol fermentation of the biomass, an inoculant comprising one or more biomass-processing biocatalyst can be added to the biomass. The inoculant can be added during chopping, as the biomass is introduced to the chamber or at any point during the chopping or chamber filling process. For example, the inoculant can comprise one or more biomass-processing biocatalyst to effect saccharification and/or alcohol fermentation of the biomass (e.g., a lignocellulose-processing enzyme, an alcohol-producing microbe, etc.). Thus, in some embodiments, the biomass is inoculated with an ethanol-producing or other alcohol-producing microbe. In some embodiments, the biomass is inoculated with both a lignocellulose-processing enzyme (i.e., a cellulose-, hemicellulose-, starch-, or lignin-degrading enzyme) and an ethanol-producing microbe. In some embodiments, the biomass comprises, at least in some portion, transgenic plant material that contains one or more lignocellulose-processing enzyme.

In one embodiment an ethanol producing biocatalyst microbe is inoculated onto chopped biomass. In some embodiments the ethanol producing biocatalyst microbe may be added to the collected biomass prior to chopping. In yet another embodiment the ethanol producing biocatalyst microbe may be added to the chamber. In some embodiments the inoculant comprises Saccharamyces cerevisiae.

In some embodiments, water soluble products and at least about 80% of the biomass-derived water is extracted from the biomass slurry to provide the biomass slurry extract. In some embodiments, at least about 85% of the biomass-derived water is extracted from the biomass slurry to provide the biomass slurry extract. In some embodiments, at least about 90% or at least about 95% of the biomass-derived water is extracted from the biomass slurry to provide the biomass slurry extract. In some aspects the biomass-derived water is extracted and recycled in a ethanol biofuel factory for re-use. In some embodiments sugars may be concentrated and stored. Furthermore, some embodiments may concentrate sugars and recycle the biomass-derived water back into the process.

In some embodiments, the biomass slurry extract comprises biomass-derived water and an alcohol. In some embodiments, the biomass slurry extract comprises biomass-derived water and fermentable sugars. In some embodiments, the biomass slurry extract comprises biomass-derived water, fermentable sugars, and an alcohol. Thus, the biomass slurry extract can have a composition that includes biomass-derived molecules that can be used in the production of biofuels without further saccharification.

Depending on the conditions within the chamber and upon the content of the biomass, the biomass slurry extract can also include additional water-soluble molecules related to the biomass treatment process. For example, the biomass slurry extract can include unfermented water-soluble carbohydrates that can require further hydrolysis prior to fermentation, as well as other fermentation products (although generally as minor constituents), including organic acids, such as acetic acid, lactic acid, butyric acid, and propionic acid.

The liquid nature of the biomass slurry extract allows for cost-effective and convenient storage and transport of useful plant-derived molecules. For example, the biomass slurry extract can be stored at the site of production or elsewhere in any suitable liquid storage container. In some embodiments, the biomass slurry extract can be transported from the treatment and extraction location to a central storage or collection facility, or directly to any suitable site for further processing (e.g., a conventional ethanol plant, either previously existing or newly built) so that the components in the biomass slurry extract can be further fermented or separated from one another. Transportation of the biomass slurry extract can be by any convenient liquid transport method, such as by truck, rail, ship (e.g., tanker, barge, etc.), air, or pipeline. In some embodiments, the alcohol in the biomass slurry extract can be separated from the other components via distillation or another drying technique (e.g., storage over sieves) to provide a purified alcohol. The purified alcohol can be denatured or mixed with other fuels (e.g. gasoline) to provide a biofuel blend, if desired.

### III. BIOMASS CONSIDERATIONS

As used herein, “biomass” particularly refers to cellulosic or lignocellulosic biomass material derived from plants, and includes material comprising cellulose and optionally further comprising hemicellulose, lignin, starch, oligosaccharides, and/or monosaccharides. Biomass can also comprise additional components, such as proteins and lipids.

Biomass can be derived from a single source or can comprise a mixture derived from more than one source. For example, biomass can comprise a mixture of material from multiple plant species, multiple hybrids or varieties of the same plant species, and multiple parts of a single plant species. Thus, the plant material can be a mixture of corn stover and various grasses, or a mixture of whole corn plant and corn stover.

In the presently disclosed processes, at least a portion of the biomass material is harvested at or in close proximity to the treatment site comprising the chamber. In some embodiments, biomass material that has not been recently harvested (e.g., paper waste or municipal solid waste) or that has been transported from a distance greater than about 8 kilometers can be added to the plant material harvested on site, so long as the costs of acquiring and transporting the added biomass material do not negatively effect the overall cost-effectiveness of the process. In some embodiments, at least 50% of the biomass is plant material harvested at or in close proximity to the chamber.

Biomass includes, but is not limited to bioenergy crops, agricultural residues, sludge from paper manufacture, yard waste, wood and forestry waste, municipal solid waste and industrial solid waste. Examples of biomass include, but are not limited to, corn grain, corn cobs, corn stover, corn silage, grasses, wheat, wheat straw, barley, barley straw, hay, rice straw, switchgrass, waste paper, sugar cane bagasse, sorghum, soy, components obtained from processing of grains, trees, branches, roots, leaves, wood chips, sawdust, shrubs and bushes, vegetables, fruits, flowers, and algae.

As used herein, the phrase “plant material” and “plant part” interchangeably refers to all or part of any plant material that comprises lignocellulose, cellulose, fermentable sugars, starches, and/or other molecules that can be broken down into fermentable sugars. The plant material can be derived from a grain, fruit, legume, seed, stalk, wood, vegetable, root, leaf tissue or a part thereof. In some embodiments, the biomass comprises plant material derived from a plant selected from one or more of the group consisting of maize (i.e., corn), soybean, millet, milo, rye, wheat, triticale, oats, barley, rice, sorghum, sudangrass, switchgrass, Miscanthus, alfalfa, cotton, sisal, hemp, jute, turf grass, rape, sunflower, willow, eucalyptus, poplar, pine, willow, tobacco, clover, bamboo, flax, pea, radish, turnip, potato, sweet potato, cassaya, taro, beet, sugar beet, sugar cane, and canola. In some embodiments, the biomass comprises one or more of the group consisting of whole plant corn, corn stover, corn cobs, and soybean forage.

The moisture content of the plant biomass can be adjusted to optimize biomass conversion conditions. In some embodiments, the moisture content of the biomass can be between about 50% and about 90%. The moisture content of plant biomass used for animal fodder silage is generally lower than about 70% to maintain feeding value, palatability, or to limit seepage that can lead to a loss of nutrients. In the present processes, however, higher moisture content can be beneficial, for example, by facilitating extraction of alcohols and water-soluble carbohydrates (WSC) (which can include both fermentable sugars and non-fermentable polysaccharides) during the extraction process, or by facilitating the reaction of lignocellulose-processing enzymes with lignocellulose in the biomass. In some embodiments, the moisture content of the plant biomass is at least about 70%. In some embodiments, the moisture content of the plant biomass is about 75%.

In some embodiments, the harvesting or collecting of the plant biomass can be timed such that the moisture content is at a desired level. For example, the plant biomass can be harvested while still green. Thus, in some embodiments, the harvesting of the plant material can be timed to provide a plant material comprising a moisture content of at least 70% (e.g., between about 70% and about 95%). In some embodiments, the harvesting is timed to provide plant biomass comprising a moisture content of at least about 75% (e.g., between about 75% and about 95%). The plant biomass can then be placed in a chamber in the absence of a drying period or within a period of time such the moisture content does not decrease by more than 5%.

In addition to moisture; the type of biomass or of the various plant components in a biomass mixture (either plant species, particular plant variety or hybrid, or plant part) can be selected based on one or more additional characteristics, including sugar content, cellulose content, lignin content, cost, length of growing season, drought resistance, disease resistance, individual plant size, and tonnage (i.e., tons of plant biomass produced/hectare). Thus, in some embodiments, a plant biomass having relatively high sugar or cellulose content can be placed into a chamber, or added to plant biomass having lower sugar or cellulose content and placed into a chamber, to optimize biomass conversion conditions (e.g., to increase the overall yield of fermentable sugars or alcohol in the biomass slurry extract, or to increase the overall biofuel yield). Plant biomass with low lignin content can be used to facilitate fuller saccharification. For example, plant materials and methods described in U.S. Patent Publication 2006/0260011 herein incorporated by reference may be used in some embodiments.

Based on the chemical or general biomass content of various segmented corn hybrids, it appears that corn plants with large amounts of stalk can be advantageous. As described herein below in the Example 4, segmented corn plant samples comprising stalk material comprise a higher percentage of fermentable dry matter than do samples comprising plant leaves, silk and husks.

Sterility is also expected to increase sugars in corn hybrids. Male sterile corn plants, such as tropical corn hybrids, without kernels, allow for sugars to accumulate in the stalks, and tend to stay greener longer. For example, recent reports describe a tall tropical maize hybrid comprises 25% or more sugar. University of Illinois at Urbana-Champaign “If Corn Is Biofuels King, Tropical Maize May Be Emperor,” ScienceDaily, Oct. 19, 2007. Thus, in some embodiments, the plant material is derived from a male sterile, tropical corn plant generally taller than 3 meters.

In some embodiments, specific corn hybrids, forage-type soybean varieties or hybrids, or varieties of other plants can be developed for use in the presently disclosed processes, wherein said hybrids and varieties maximize metric tons/hectare or convertible sugars/hectare. In some embodiments, a uniform biomass mixture can be developed to optimize the conversion of biomass to sugar and/or alcohol, thereby providing maximal use of biomass-producing acreage.

In some embodiments, harvesting the biomass can comprise harvesting the total “aerial biomass,” i.e., all the biomass material of a plant growing above the soil surface. In some embodiments, the height of cut of the plant can be varied to affect the saccharification and/or alcohol fermentation of the plant material. In particular, cut height can be varied because of changes in the lignocellulose content of plant material closer to the soil surface. Many plants have higher lignin and store nitrates in the first few centimeters above the soil surface. For example, a normal silage cutter having a cut height of about 15 centimeters can be used to harvest the biomass. The cut height can also be greater than about 15 centimeters or can be lower than 15 centimeters, depending upon the biomass lignin content, field erosion standards, or other parameters.

Seasonal considerations can be taken into account when choosing a biomass source. Plant biomass can be varied based on seasonal availability. For example, spring cool season plants (e.g., field peas, oats, wheat, barley, etc.) produce the most biomass in the spring, whereas warm season plants (e.g., corn, sorghum, millet, soybeans, etc.) produce the most during the summer. Some cool season plants (e.g., beets, turnips, etc.) accumulate the most biomass in the fall. Biomass harvest can be determined by processing needs and optimum biomass accumulation. With rapid turnover, multiple-cut biomass harvests can occur throughout the growing season. Fast regrowing crops include, but are not limited to forage sorghum, sudangrass, pearl millet, and alfalfa. These crops and other cool season crops (e.g., field pea, forage radish, turnips, beets, rye winter wheat, and winter canola) can be rotated through the presently disclosed systems, thereby maximizing tons harvested and providing crop rotation.

In some embodiments, sugar cane tops normally discarded as waste or left in the field, may be collected wherein the material contains approximately greater than or equal to 60% moisture. Once collected these sugar cane tops can then be chopped, inoculated, added to the chamber and fermented as described herein.

In some embodiments, the biomass comprises plant material from a transgenic plant. In some embodiments, the transgenic plant is engineered to produce one or more proteins that facilitate the processing of the biomass to biofuel. In some embodiments, the transgenic plant is a plant that has been engineered to produce one or more lignocellulose-processing enzyme. Types of lignocellulose-processing enzymes include, but are not limited to, cellulases, hemicellulases, ligninases, and starch-degrading enzymes, such as amylases. For example, the genome of the transgenic plant can be augmented with a recombinant polynucleotide encoding at least one lignocellulose-processing enzyme (e.g., an amylase) operably linked to a promoter sequence, wherein the polynucleotide sequence is optimized for expression in the plant. The lignocellulose-processing enzyme can be expressed constitutively or tissue-specifically (for example in leaves and stems). In some embodiments, the transgenic plant is engineered to produce an enzyme (i.e., an isomerase) for converting one sugar into another, more easily fermentable sugar. In some embodiments, the transgenic plant is engineered to produce less lignin or more cellulose.

In some embodiments, the biomass can comprise plant material from a plurality of transgenic plants, wherein each of the plurality of transgenic plants produces a different lignocellulose-processing enzyme. In some embodiments, the biomass comprises plant material from a single transgenic plant that produces several different lignocellulose-processing enzymes. In some embodiments, the biomass comprises a mixture of transgenic plant material and non-transgenic plant material. Thus, the transgenic plant material can be used as an enzyme source for the processing of both the transgenic plant material and non-transgenic plant material, thereby reducing or eliminating the need to add enzyme from another source.

Delivery or introduction of a nucleic acid construct into a plant cell to provide a transgenic plant can be accomplished using a variety of methods known in the art. Suitable methods include non-biological methods, such as microinjection, microprojectile bombardment, electroporation, induced uptake, and aerosol beam injection, as well as biological methods, such as direct DNA uptake, liposomes, and Agrobacterium-mediated transformation. U.S. Patent Application Publication Nos. 2007/0250961 and 2002/0138878, and PCT International Publication WO 98/16651, each of which is incorporated by reference herein in its entirety, describe methods for transforming plants, as well as a variety of suitable genes related to lignocellulose-processing enzymes.

The processing of collected plant biomass that occurs prior to introduction into the chamber can also be manipulated to affect the outcome of saccharification and/or fermentation processes. Plant biomass can be reduced in size to increase packing of the materials, providing for a quicker conversion to anaerobic conditions, or to increase the surface area of the plant biomass for enzymatic action. Thus, in some embodiments, the biomass can be ground or cut prior to being placed into the chamber, using, for example, a conventional silage chopper. The theoretical length of cut (TLC) of the chopped or ground biomass can be varied to optimize microbial or enzymatic conditions within the chamber.

In some embodiments, the TLC of the chopped biomass can be between about 0.3 centimeters and about 1.3 centimeters. Additionally, freezing and thawing the biomass material just prior to being placed into the chamber can aid in breaking down the lignocellulose therein and/or to arrest endogenous microbes. The biomass material could be frozen, for example, using liquid or solid CO₂ prepared from CO₂ gas collected as a co-product of biomass conversion process. Biomass collecting can also be timed to follow a freeze.

### IV. INOCULANTS AND CONDITIONS

IV.A. Biomass-Processing Biocatalysts

In some embodiments, one or more biomass-processing biocatalyst is added to the biomass as part of an inoculant, for facilitating the saccharification and/or alcohol fermentation of the biomass. The term “biomass-processing biocatalyst” refers to enzymes and microbes (e.g., bacteria, fungi, archaea, or protists) that degrade or convert biomass-derived molecules. For example, the biomass-processing biocatalyst can be a lignocellulose-processing enzyme or an alcohol-producing microbe. In some embodiments, one or more biomass-processing biocatalyst is applied to the growing biomass prior to collection (e.g. ground or aerial application, or by irrigation).

The term “biomass-processing enzyme” refers to enzymes that degrade or convert biomass derived molecules. Suitable biomass processing enzymes include, but are not limited to α-amylase, endo or exo-1,4, or 1,6-α-D, glucoamylase, glucose isomerase, β-amylases, α-glucosidases, and other exo-amylases; and starch debranching enzymes, such as isoamylase, pullulanase, neo-pullulanase, iso-pullulanase, amylopullulanase and the like, glycosyl transferases such as cyclodextrin glycosyltransferase and the like, cellulases such as exo-1,4-β-cellobiohydrolase, exo-1,3-O-D-glucanase, hemicellulase, β-glucosidase and the like; endoglucanases such as endo-1,3-β-glucanase and endo-1,4-β-glucanase and the like; L-arabinases, such as endo-1,5-α-L-arabinase, α-arabinosidases and the like; galactanases such as endo-1,4-β-D-galactanase, endo-1,3-β-D-galactanase, 1-galactosidase, α-galactosidase and the like; mannanases, such as endo-1,4-β-D-mannanase, β-mannosidase, α-mannosidase and the like; xylanases, such as endo-1,4-1-xylanase, β-D-xylosidase, 1,3-β-3-D-xylanase, and the like; and pectinases; and non-starch processing enzymes, including protease, glucanase, xylanase, thioredoxin/thioredoxin reductase, esterase, phytase, and lipase.

In one embodiment, the biomass processing enzyme is a starch-degrading enzyme selected from the group, but not limited to for example α-amylase, pullulanase, α-glucosidase, glucoamylase, amylopullulanase, glucose isomerase, or combinations thereof.

In another embodiment, the biomass processing enzyme is a non-starch processing enzyme selected from the group but not limited to for example cellulases, protease, glucanase, xylanase, and esterase.

Combinations of biomass processing enzymes are further envisioned by the present invention. For example, starch-processing and non-starch processing enzymes may be used in combination. Another method includes the use of exogenous enzyme(s). Herein, the phrase “exogenous addition of enzyme” or “exogenously added enzyme” interchangeably refers to the addition of a enzyme that is, for example, the liquid addition of commercially available enzymes.

The terms “lignocellulytic enzyme” and “lignocellulose-processing enzyme” refer to biomass processing enzymes that are involved in the disruption and or degradation of lignocelluloses. The disruption of lignocellulose by lignocellulytic enzymes leads to the formation of substances including monosaccharides, disaccharides, polysaccharides and phenols. Lignocellulytic enzymes include, but are not limited to, cellulases, hemicellulases, amylases, and ligninases. Thus, lignocellulytic enzymes include saccharification enzymes, i.e., enzymes which hydrolyze polysaccharides. Saccharification enzymes and their use in biomass treatments have been previously reviewed. See Lynd, L. R., et al., Microbiol. Biol. Rev., 66, 506-577 (2002).

Cellulases are enzymes involved in cellulose degradation. Cellulase enzymes are classified on the basis of their mode of action. There are two basic kinds of cellulases: the endocellulases, which cleave polysaccharide polymer chains internally; and the exocellulases, which cleave from the reducing and non-reducing ends of molecules generated by the action of endocellulases. Cellulases include cellobiohydrolases, endoglucanases, and β-D-glucosidases. Endoglucanases randomly attack the amorphous regions of cellulose substrates, yielding mainly higher oligomers. Cellulobiohydrolases are exocellulases which hydrolyze crystalline cellulose and release cellobiose (glucose dimer). Both types of enzymes hydrolyze-1,4-glycosidic bonds. β-D-glucosidases or cellulobiase converts oligosaccharides and cellubiose to glucose.

Thus, in some embodiments, the biocatalyst is cellulase (E.C. 3.2.1.4), also known as an endoglucanase, which catalyzes the hydrolysis of 1,4-β-D-glycosidic linkages. The cellulase can be of microbial origin, such as derivable from a strain of a filamentous fungus (e.g., Aspergillus, Trichoderma, Humicola, Fusarium). Commercially available cellulase preparations which can be used include, but are not limited to, CELLUCLAST™, CELLUZYME™, CEREFLO™, and ULTRAFLO™ (available from Novozymes A/S, Bagsvaerd, Denmark), SPEZYME™ CE and SPEZYME™ CP (available from Genencor International, Inc., Palo Alto, Calif., United States of America) and ROHAMENT® CL (available from AB Enzymes GmbH, Darmstadt, Germany). In some embodiments commercially available enzyme preparations as those described above can be added to the biomass exogenously prior to biomass being placed in the chamber. In some embodiments exogenous enzyme preparations may be added to the chamber and mixed into the biomass.

Hemicellulases are enzymes that are involved in hemicellulose degradation. Hemicellulases include xylanases, arabinofuranosidases, acetyl xylan esterases, glucuronidases, mannanases, galactanases, and arabinases. Similar to cellulase enzymes, hemicellulases are classified on the basis of their mode of action: the endo-acting hemicellulases attack internal bonds within the polysaccharide chain; the exo-acting hemicellulases act progressively from either the reducing or non-reducing end of polysaccharide chains. More particularly, endo-acting hemicellulases include, but are not limited to, endoarabinanase, endoarabinogalactanase, endoglucanase, endomannanase, endoxylanase, and feraxan endoxylanase. Examples of exo-acting hemicellulases include, but are not limited to, α-L-arabinosidase, β-L-arabinosidase, α-1,2-L-fucosidase, α-D-galactosidase, β-D-galactosidase, β-D-glucosidase, β-D-glucuronidase, β-D-mannosidase, β-D-xylosidase, exo-glucosidase, exo-cellobiohydrolase, exo-mannobiohydrolase, exo-mannanase, exo-xylanase, xylan α-glucuronidase, and coniferin β-glucosidase.

Ligninases are enzymes that are involved in the degradation of lignin. A variety of fungi and bacteria produce ligninases. Lignin-degrading enzymes include, but are not limited to, lignin peroxidases, manganese-dependent peroxidases, hybrid peroxidases (which exhibit combined properties of lignin peroxidases and manganese-dependent peroxidases), and laccases. Hydrogen peroxide, required as a co-substrate by the peroxidases, can be generated by glucose oxidase, aryl alcohol oxidase, and/or lignin peroxidase-activated glyoxal oxidase.

In addition to cellulases, hemicellulases and ligninases, lignocellulolytic enzymes that can be used in the practice of the presently disclosed subject matter also include enzymes that degrade pectic substances. Pectic substances are composed of homogalacturonan (or pectin), rhamnogalacturonan, and xylogalacturonan. Enzymes that degrade homogalacturonan include pectate lyase, pectin lyase, polygalacturonase, pectin acetyl esterase, and pectin methyl esterase. Enzymes that degrade rhamnogalacturonan include α-arabinofuranosidase, β-galactosidase, galactanase, arabinanase, α-arabinofuranosidase, rhamnogalacturonase, rhamnogalacturonan lyase, and rhamnogalacturonan acetyl esterase. Enzymes that degrade xylogalacturonan include xylogalacturonosidase, xylogalacturonase, and rhamnogalacturonan lyase. Other enzymes that may enhance or promote lignocellulose disruption and/or degradation include, but are not limited to, esterases, lipases, phospholipases, phytases, proteases, and peroxidases.

Fermentation of fermentable sugars to ethanol can be carried out by one or more appropriate biocatalysts (e.g., enzymes and/or microbes). Suitable ethanol-producing biocatalysts can be micro-organisms selected from bacteria, filamentous fungi, and yeast. The ethanol-producing biocatalyst can be a wild type micro-organism, a mutated micro-organism, or a recombinant micro-organism and can include, for example, Escherichia, Zymomonas, Saccharomyces, Candida, Pichia, Streptomyces, Bacillus, Lactobacillus, and Clostridium. In some embodiments, the ethanol-producing biocatalyst can be selected from the group consisting of recombinant Escherichia coli, Zymomonas mobilis, Bacillus stearothermophilus, Saccharomyces cerevisiae, Clostridia thermocellum, Thermoanaerobacterium saccharolyticum, and Pichia stipitis. In some embodiments, the fermentation or other biomass-processing microbe of the presently disclosed processes is an anaerobic microbe.

Biocatalysts for fermentation to produce ethanol include those that have been previously described, or those that can be discovered, produced through mutation, or engineered through recombinant means. In particular, U.S. Patent Application Publication No. 2007/0178569, incorporated herein by reference in its entirety, describes Clostridium phytofermentans, an anaerobic bacterium that can ferment cellulosic material to fuel (e.g., ethanol) directly, without another chemical or enzymatic treatment. Other suitable ethanol-producing organisms include those which can utilize carbon monoxide from biomass to produce ethanol, including Butyribacterium methylotropicum, Clostridium autoethanogenum, Clostridium carboxidivorans, and Clostridium ljungdahlii. See, e.g., U.S. Patent Application Publication No. 2007/0275447, incorporated herein by reference in its entirety, with regard to Clostridium carboxidivorans.

Biomass-processing biocatalysts generally have or can be adapted to have a preferred temperature and pH range for activity. In some embodiments, the biocatalysts can (or can be adapted to) operate at pH ranges between 4 and 5. In some embodiments, the biocatalysts can operate at temperatures ranging from ambient temperature to about 45° C. above ambient temperatures. In some embodiments, the optimal pH and/or temperature ranges (the ranges that give maximal activity) of the saccharification and/or fermentation enzymes used as inoculant can be specifically selected so that acid fermentation products (e.g., lactic acid, acetic acid) are minimized. For example, an enzyme can be selected to have an optimal pH range that does not overlap with that of lactic acid fermentation enzymes that can be present in the chamber. In some embodiments, the enzymes or microbes can be selected to function under high alcohol and/or sugar concentrations. In some embodiments, the enzymes or microbes can be selected for optimal activity under temperature conditions present in the chamber.

IV.B. Other Additives

A number of other inoculant components can be added to the biomass, either in combination with a biomass-processing biocatalyst or alone. While these additives do not directly saccharify or ferment the biomass to alcohols, they can be added to control the rate of saccharification or fermentation, or to control the ratio of products expected in the biomass slurry extract. In some embodiments, a biomass-processing biocatalyst is used in combination with other inoculant additives to provide an optimum environment for the biocatalyst.

In some embodiments, one or more inoculant component is added as a nutrient or growth factor for one or more biomass-processing biocatalyst present in the biomass prior to harvest, as harvested, or intentionally added. For example, the inoculant can include one or more growth factor or nutrient, such as a vitamin or mineral. Vitamins include, but are not limited to, biotin, folic acid, pyridoxine, riboflavin, urea, yeast extract, thymine, tryptone, adenine, cytosine, guanosine, uracil, nicotinic acid, pantothenic acid, B12 (cyanocobalamin), and p-aminobenzoic acid. Minerals can include, but are not limited to, MgSO₄, MnSO₄, FeSO₄, CaCl₂, CoCl₂, ZnCl₂, CuSO₄, AlK(SO₄)₂, H₃BO₃, Na₂MoO₄, NiCl₂, NaWo₄, and hydrates thereof.

In some embodiments, the inoculant includes one or more chelator or surfactant. In some embodiments, the inoculant includes a softener or plasticizer. The inoculant can also include some solid material as a filler or extender to help mix the inoculant components into a uniform composition and/or to help spread the inoculant in the biomass evenly.

In some embodiments, the inoculant includes a pH-adjusting agent to raise or lower the pH of the liquefying biomass or to help maintain the pH in a desired range. The pH-adjusting agent can be an acid, a base, a buffering agent, or combinations thereof. Thus, in some embodiments, the inoculant can include one or more pH-adjusting agent such that the pH within the chamber can be adjusted or maintained under optimal conditions for the lignocellulytic and/or alcohol fermentation enzymes and microbes present or added to the biomass.

The inoculant can also include an electrolyte, such as NaCl or KCl. The electrolyte can be used to control the water activity of the liquids within the biomass.

In some embodiments, the inoculant can include one or more nitrogen-containing agents, such as, ammonia, ammonium hydroxide, ammonium chloride, urea, ammonium nitrate, or ammonium phosphate. The nitrogen-containing agent can act as a nutrient, a pH-adjusting agent, an oxygen-depleting agent, or to add nutritional value to the residual solids produced during the process.

In some embodiments, the inoculant can contain an antimicrobial agent directed to inhibit the activity of an undesirable microbe present in the chamber. For example, the inoculant can comprise an antibacterial agent to inhibit the activity of a lactic acid bacteria or any other microbe that utilizes fermentable sugars for a purpose other than alcohol production.

In some embodiments, the inoculant can comprise one or more oxygen-depleting agent, used to facilitate the achievement of an anaerobic environment in the chamber. Solid oxygen-depleting agents include, but are not limited to, chloropicrin. In some embodiments, the oxygen-depleting agent is a gas, such as CO₂, N₂, or H₂. The gas can be produced by a microbe or by enzymatic action within the chamber, or the gas can be specifically added to the chamber directly. Thus, in some embodiments, an oxygen-depleting gas such as CO₂ is added to the chamber during or immediately after filling with the biomass, displacing some or all of the O₂ gas present.

In some embodiments, at least a portion of the inoculating material can be added during biomass chopping to facilitate even mixing of the inoculant. In some embodiments, one or more biomass-processing biocatalyst can be added to the plant biomass during the chopping. In some embodiments, one or more pH-adjusting agent, nitrogen-containing agent, or nutrient is added to the plant material during chopping. In some embodiments, at least a portion of the inoculant is added while the plant biomass is being placed into the chamber. Additionally, the chamber can be adapted so that inoculant can be added at any time after the plant biomass has been originally added into the chamber, to adjust the saccharification and/or fermentation rate as necessary. For example, additional biocatalyst or pH-adjusting agents can be added at any time after the plant biomass has been introduced into the chamber to adjust conditions to promote desired saccharification or fermentation or to reduce undesirable fermentation reactions.

IV.C. Chamber Conditions and Monitoring

When anaerobic biocatalysts are employed, conditions within the chamber can be controlled to achieve anaerobic conditions as rapidly as possible. As noted above, oxygen-depleting agents can be added, before, during, or after the filling of the chamber with the biomass. Other factors, such as length of cut, biomass compaction, moisture content, and/or initial sugar content can be used to speed achievement of anaerobic conditions, as well.

The providing and placing of the biomass into the chamber is usually performed in the absence of added water (or with the addition of only a minimal amount of water used to provide an inoculant solution that can be sprayed onto the biomass). Thus, in some embodiments, the presently disclosed processes provide saccharification and/or fermentation with biomass-derived water as the sole liquid medium. In some embodiments, the biomass-derived water accounts for at least about 90%, at least about 95%, or at least about 98% of the water present in the chamber.

Under conventional lactic acid fermentation ensiling conditions, the temperature of ensiled biomaterials, such as corn stover, can be about 15-20° C. above ambient temperature (i.e., the temperature of the plant material prior to being ensiled). According to the presently disclosed processes, heat inside the chamber can be controlled by length of chop, moisture content, sugar content, enzyme load, packing system, natural microbe control, the addition of non-plant nitrogen, the size (and/or depth) of the container, inoculation with beneficial microbes, and with heat exchange or cooling systems. For example, increased moisture content and shorter cut length can combine to increase heat in the chamber above that typically observed during the ensiling of plant biomass for use as fodder, when high heat is undesirable because it can cause a loss of nutrient value. In some embodiments higher heat levels can be desired to decrease endogenous microbes and/or to optimize efficiency of inoculations suitable for higher temperatures, such as enzymes developed to be active at higher temperatures (i.e., greater than about 50° C. or about 60° C.).

In some embodiments, conditions within the chamber can be controlled so that temperatures reach between about 25-49° C. above the temperature outside of the chamber. The heat can be used to increase the conversion of biomass to sugars and ethanol, in the absence of heat added from an outside source. By using the heat generated by the conditions in the chamber, the presently disclosed processes can reduce costs generally associated with biomass processing that involves the addition of heat to biomass/saccharification enzyme slurries and fermentation baths.

During the saccharification or saccharification/alcohol fermentation, the biomass can be monitored to determine when to collect the biomass slurry. Based on monitoring data, it can also be determined that additional inoculant be added to the chamber or that the conditions be altered in some other manner to increase saccharification and/or alcohol production or to decrease acid fermentation.

Thus, in some embodiments, the placing of the biomass into the chamber comprises filling the chamber with biomass; and monitoring one or more of temperature, atmospheric oxygen level, escaping gases, pH, production of saccharification products, production of alcohol fermentation products, and production of acid fermentation products.

Based on the presence or relative concentrations of acid fermentation products to saccharification products and alcohols, pH-adjusting agents and/or additional biocatalyst inoculants can be added to the biomass mixture. For example, the presence of high levels of lactic, acetic, or butyric acid can indicate that fermentable sugars are being used in alternative fermentation processes, and correction of the conditions within the chamber can be performed. The pH can be adjusted to favor alcohol fermentation biocatalysts or additional alcohol biocatalysts can be added to the chamber.

Conversion of the biomass will usually take at least about 20 hours, but could also take several days or months. In some embodiments, the period of time is between about 20 hours to about 21 days. In some embodiments, the period of time is between about 4 hours to about 12 hours. In some embodiments, the period of time is between about 12 hours to about 24 hours. In some embodiments, the period of time is between about 24 hours to about 72 hours. In some embodiments, the period of time is between about 24 hours and about 48 hours or between about 24 hours and about 36 hours. The period of time can vary depending on factors including, but not limited to, the type of biomass used, the amount of biomass used, moisture of incoming biomass, the desired composition of the biomass slurry extract, the amount of biocatalysts present, temperature, and pH.

IV.D. Analytical Methods

By “dry matter” or “dry weight” of biomass is meant the weight of the biomass having all or essentially all water removed. Dry matter (DM) can be measured according to American Society of Testing and Materials (ASTM) Standard E1756-01 (Standard Test Method for Determination of Total Solids in Biomass). DM of paper-related biomass can be determined via Technical Association of the Pulp and Paper Industry, Inc (TAPPI) Standard T-412 om-02 (Moisture in Pulp, Paper, and Paperboard).

The moisture content of plant material can be tested according to a variety of methods known in the art. For example, the moisture content of plant material can be calculated based upon the weight lost during drying of the plant material (e.g., the difference between the weight of the raw biomass and the biomass DM, i.e., 1−DM or 100%−% DM). Moisture content of plant material can also be tested using commercially available moisture meters.

Dry chemistry analysis of DM, ash, neutral detergent fiber (NDF) (e.g., lignin, hemicellulose, and cellulose), acid detergent fiber (ADF), lignin, and crude protein content can be performed, for example, by near infrared reflectance spectroscopy (N IRS). Other established feed analysis procedures can also be used to analyze biomass feedstocks and saccharified biomass to determine levels of neutral detergent fiber (NDF), NDF digestibility, and non-structural carbohydrates (NSC). See, e.g., Chen, Y., et al., Appl. Biochem. Biotechnol., 143, 80-92 (2007).

Soluble sugars (e.g., glucose, cellobiose, xylose, galactose, arabinose, mannose, etc.), acetamide, lactic acid, and acetic acid present in biomass liquid extracts can be measured via HPLC. For more information concerning suitable HPLC methods for the determination of carbohydrates, soluble sugars and other water soluble components, see U.S. Patent Application Publication No. 2007/0031953. Water-soluble carbohydrate can also be determined using the phenol sulfuric acid method, while alcohol content can be determined using gas chromatography. See Pedroso, et al., Sci. Agric., 62(5), 427-432, 2005. Ethanol content can also be analyzed via enzymatic assays using alcohol dehydrogenase, described, for example, in Chen, Y., et al., Appl. Biochem. Biotechnol., 143, 80-92 (2007).

The pH of the biomass within the chamber can be determined by shaking a small sample of biomass removed from the chamber with water for a minute or two and analyzing the pH of the water with commercially available pH-sensitive paper. Alternatively, a pH meter can be used for more accurate readings.

### V. EXTRACTION

Methods for the extraction of the biomass-derived water and water soluble biomass products (i.e. biomass slurry extract) from the biomass slurry include, but are not limited to, decanting, filtering (including vacuum filtering), pressing, centrifuging, and other solid-liquid extraction methods. Thus, in some embodiments, the extracting comprises one or more of centrifuging the biomass slurry, pressing the biomass slurry, and decanting the biomass slurry. In some embodiments, the biomass slurry is serially extracted, in a portion-wise manner. If serial extraction is employed, portions of biomass slurry are unloaded from the bottom of the chamber and extracted, one at a time, until all of the biomass slurry is extracted, or until a desired amount of the biomass slurry is extracted.

While the majority of the liquids present in the biomass are extracted to provide the biomass slurry extract, extraction is not always exhaustive. In some embodiments, the biomass slurry extract comprises about 80% of the biomass-derived water from the biomass. The majority of the water soluble molecules present in the biomass slurry can be dissolved in the biomass-derived water. Thus, the biomass slurry extract can also comprise fermentable sugars and alcohol. In some embodiments, the biomass slurry extract can be concentrated to increase concentrations of desired molecules. In some embodiments, the biomass slurry extract can comprise up to about 90% alcohol. In some embodiments, the biomass slurry extract can comprise between about 1% and about 90% alcohol.

The residual solids fraction can comprise about 20% of the plant-derived water, as well as the non-water soluble molecules from the biomass slurry, including lignins and unhydrolyzed cellulose and hemicellulose. The residual solids fraction of the biomass can be washed with water to remove additional adsorbed sugars, if desired, following any previously performed extraction step.

The biomass slurry extract can be stored for a time in a suitable liquid storage facility prior to transportation to a second location for further processing for the production and/or purification of the biofuel. Following extraction, the pH of the biomass slurry extract can be adjusted to better facilitate storage or transport. For example, when the pH of the biomass slurry extract is relatively acidic (e.g., less than about 6 or less than about 5), it can be adjusted to a neutral range (i.e., between about 6 and about 8, or between about 6.7 and about 7.6) so as to be less corrosive to various materials used in the construction of liquid storage containers, tanker trucks or pipelines. In addition, additional enzymes and/or microbes can be added to the biomass slurry extract to affect additional saccharification, alcohol fermentation, and/or to control the viscosity of the extract (e.g., to halt gelling of any remaining polysaccharides).

### VI. PROCESS MACHINERY

The processes and systems of the presently disclosed subject matter can make use of currently available commercial harvesting equipment (e.g., forage harvesters), silage choppers, silos, silo loading and unloading equipment, extraction equipment and pumping equipment. Extraction of the biomass slurry can also take advantage of screening machinery, centrifuges, decanters, concentrators, and other extracting machinery (e.g., countercurrent extractors, screw-conveyor extractors, or vacuum-belt extractors) presently used in the ethanol production. Suitable equipment can be provided, for example, from Westfalia Separator, Inc. (Northvale, N.J., United States of America), Louisiana Chemical Equipment Co., L.P., (Baton Rouge, La., United States of America), and TM Industrial Supply, Inc. (Erie, Pa., United States of America).

The chamber of the presently disclosed processes can include any suitable container that allows for control of the atmospheric conditions within and which is made of a material that will not be affected by the enclosed piled biomass or by any added inoculants. The chamber can be a vertical silo or a horizontal silo (e.g., a bunker silo). Possible chambers also include polybags, fuel storage tanks, and lined lagoons. Suitable silos include, for example, bulk material SH-type enameled storage tanks manufactured by Vitkovice-Power Engineering, Ltd. (Ostrava, Czech Republic), or equipment commercially available from Nebraska Harvestore Systems, Inc. (Norfolk, Nebr., United States of America).

The chamber can include loading and unloading equipment (e.g., conveyors, hoppers, etc.), as well as equipment for monitoring conditions (e.g. temperature) or the contents of the chamber. The chamber can also include mixing equipment, fans, cooling equipment, and gas tanks and gas inlets and/or outlets for introducing a gas into the chamber or collecting gas from the chamber. In some embodiments, the chamber can have a sloped floor, to aid in collection of liquid seepage or in biomass extraction.

### VII. BIOMASS LIQUID EXTRACT TREATMENT FACILITIES

Biomass slurry treatment facilities of the presently disclosed subject matter can include conventional ethanol-producing facilities for use in preparing biofuels from corn grain and/or other lignocellulosic biomass. The treatment facilities can also be facilities specifically built and designed to process biomass slurry or pre-existing facilities.

At the treatment facility, separation of the constituents of the biomass slurry, if desired, can be done using a variety of chemical and physical techniques that rely on the different chemical and physical properties of the molecules present (e.g., sugars, organic acids, and phenols). Such techniques, include, but are not limited to, chromatography (e.g., ion exchange, affinity, hydrophobic, size exclusion), electrophoresis procedures, differential solubility, distillation and/or extraction (solid-phase or liquid-liquid).

In some embodiments, the biomass slurry comprises plant-derived water and fermentable sugars. Upon arrival of the biomass slurry at the treatment facility, the biomass slurry may be separated into the biomass slurry extract and residual solid. The biomass slurry extract can be fermented to convert the fermentable sugars present into alcohol. The fermentation can be performed using any suitable alcohol-producing biocatalyst. In some embodiments, the biocatalyst is an alcohol-producing microbe, such as, yeast. If necessary, yeast nutrients or other microbe nutrients can be added to the biomass slurry extract. The fermenting mixture can provide its own heat or the heat can be artificially maintained at a suitable temperature, i.e., between about 25-45° C., for a period of time sufficient to effect the desired amount of fermentation. When convenient, heating needs can be supplied by burning co-products of the presently disclosed processes, such as residual solids. Following fermentation, the ethanol in the fermented biomass slurry extract can be purified according to any suitable technique.

In some embodiments, the biomass slurry comprises at least some amount of ethanol. In some embodiments, the biomass slurry can comprise between about 1% and about 90% ethanol. The ethanol can be isolated from the biomass slurry or from a further fermented biomass slurry extract using methods such as distillation, azeotropic distillation, liquid-liquid extraction, adsorption, gas stripping, membrane evaporation, pervaporation, and the like.

In some embodiments, the plant-derived water from the biomass slurry is separated and recycled. The plant-derived water can be used to provide at least a portion of the water needs of the treatment facility. For example, if the facility is also used to process raw biomass feedstocks, the water can be used to dilute the raw biomass feedstock for saccharification or fermentation. The water can be used to further dilute biomass slurry extracts prior to a fermentation or distillation step. The water can be used as a coolant in distillation columns. Alternatively, the water can be piped or trucked away from the facility and used elsewhere. In some embodiments, the plant-derived water can be used to irrigate crops for use as biofuel biomass feedstock. In some embodiments, the same pipeline network used to provide the biomass slurry to the treatment facility can be used to return biomass-derived water to the harvesting site.

Average corn silage having a yield of 77.6 metric tons/hectare and a moisture content of 65.3% can produce a water yield of about 51,000 kilograms per hectare or about 53,200 liters per hectare. By increasing the corn moisture level to 75%, allowing a 5% loss before saccharification or saccharification/alcohol fermentation, the amount of available biomass-derived water increases to about 57,000 liters per hectare. Thus, the presently disclosed processes can facilitate the capture and reuse of large amounts of water.

### VIII. CO-PRODUCTS

In addition to bioalcohols, the presently disclosed processes can also produce several additional economically useful co-products.

The solid residuals fraction of the biomass slurry can comprise lignin, protein, unhydrolyzed and/or partially hydrolyzed lignocellulose, and a variety of other components, including some of the biomass-derived water and water soluble sugars. The solid residuals fraction can comprise useful nutrients, including, but not limited to, nitrogen, phosphorous, potassium and others, which can be useful in feeds and fertilizers. Thus, in some embodiments, the solid residuals faction can be dried to provide an animal feed or a fertilizer that can be used, for example, on fields where biomass is being produced (e.g., to reintroduce unfermented lignin and other constituents to replenish or build soil organic matter). In some embodiments, the solid residuals can be burned to provide fuel for boilers. In some embodiments, the solid residuals can be used to provide methanol.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Test Scale Preparation and Extraction of Biomass Slurry

NK Brand hybrid N40T corn was grown by planting seeds in Sioux Falls S. Dak. on sandy-clay-loam ground irrigated occasionally to maintain soil moisture. Seeds germinated and the subsequent plants were allowed to mature. Prior to pollination, developing ears were removed. Removing ears prior to pollination is a known method in the art to increase plant sugars (i.e. sucrose, fructose and glucose). Subsequent ears were also removed on the lower nodes to prevent kernel development. Controls where ears were not removed consisted of the same hybrid planted in the same or adjacent rows to corn having ears removed prior to pollination. Controls containing ears were in late dough/early dent (approximately 10 dents showing per ear) when harvested; however, the ear was removed at harvest and was not included in the fermentation studies described herein.

Corn plants were harvested by cutting the plants approximately 6 inches above the soil surface and placing the whole plant in a large black plastic bag. Harvested plant biomass was placed into a fermentation chamber within 1 to 2 hours of harvest. Depending on the source material to be placed in the fermentation chamber, the time from harvest to placing biomass into the chamber should not exceed 24 hours and more preferably occurs within about 5 hours and most preferably prior to 1 hour.

Yeast inoculation for fermentation experiments were generated by placing three packs of SAFBREW T-58 dry Saccharomyces cerevisiae brewing yeast (Fermentis Lesaffre Group, France) in 100 grams of distilled water and stirred to create a consistent paste mixture which thickened over time. Yeast was then smeared onto external leaves and stalks. Following inoculation with yeast, plant biomass was then chopped using a 500 watt wide mouth food processor (Black & Decker Power Pro, Maryland). The first chopping pass used a slicing blade which cut approximately 0.3 cm but left many large fibrous strands and some long longitudinal slices. A second chopping pass used a double spinning chopping blade attachment which cut most of the slices into smaller pieces generally 0.5 cm or less. Any remaining longer pieces and fibrous strands were cut with scissors to 0.5 inches or less. The yeast inoculated samples were chopped and placed in a large steel bowl for further mixing.

A control fermentation was also generated which was not inoculated with yeast.

Chopped bagged samples were allowed to ferment by placing in a atmospherically controlled chamber pre-heated to 26° C. with temperature allowed to rise and fall to simulate conditions one would expect to measure in a silo. Control samples were created that were not allowed to ferment by placing in a refrigerator and storing at 7.1° C.

After approximately 1 hour, all yeast inoculated sample bags were filled with air. Air was extracted with a manual pump and sample temperatures recorded. This was replicated over the course of approximately 48 hours as needed to remove air from the fermentations. Samples from the inoculated sample bags (the ferment) were collected for further analysis at approximately 12, 24, 36 and 48 hour time points. Temperatures ranged from 26° C. to approximately 38° C. over the course of the experiment. Little to no swelling was noticed in the bags that were not inoculated with yeast. Each sample was given a unique I.D. sequence. Table 1 is a key code to describe each experimental sample by using its unique I.D. sequence.

TABLE 1
Code of Letters and Number in Sample I.D. Sequence

| Character | Description |
|---|---|
| 1st letter | Product |
| 2nd letter | Testing procedure |
| 1st number | Sample number |
| 2 & 3rd numbers | Hours tested |
| last letter | Form tested (S = Solid; L = Liquid) |

Code of letters and numbers in sequence:
T = NK N40T corn; whole plant; ear pollinated and removed before testing
U = NK N40T corn; whole plant; ear removed before pollination
W = NK N40T corn stalk only from corn with ear removed just before chopping
X = Sucrosorgo 405 from Sorghum Partners; Hybrid forage sorghum; whole plant
Y = Derry forage soybeans a USDA variety; whole plant
A = Refrigerated after chopping; no yeast added if fermented
B = Yeast inoculated before chopping
Sample number followed by fermentation in hours
L = Tested in liquid form
S = Tested in solid form

Liquid was removed from the fermentations at 0, 12, 24, and 48 hours. All samples with liquids extracted were done with a heavy duty manual wheat grass Juicer (Weston Brand Model #36-3701W) with the solids throat discharge completely open to exude any remaining solids. Liquid and chopped samples were placed in quart size vacuum bags with air removed and then each sample's weight was recorded for initial weight data. The amount of liquid extracted was measured as a percentage of total sample weight.

TABLE 2
Percent Liquid Extracted From Total Sample Weight

| | Percent liquid extracted |
|---|---|
| Sample Number | (from raw sample weight) |
| UA200L | 70.1% |
| UA124L | 72.4% |
| UA148L | 73.4% |
| UB212L | 71.7% |
| UB224L | 68.9% |
| UB236L | 73.8% |
| UB248L | 72.9% |
| WA200L | 80.2% |
| TA200L | 71.6% |
| XA200L | 70.5% |
| YA200L | 48.1% |
| UA400L | 70.4% |
| UC348L | 76.6% |
| TA400L | 71.6% |

This data demonstrates that over 70% of the volume of plant biomass can be extracted as a liquid. Samples comprising stalk only (WA200L) had the highest liquid extraction percent at 80.2% followed by yeast inoculated fermentation sample (UC348L) which had a percent liquid extraction of 76.6%. The data shows that over a relatively short amount of time, fresh biomass containing approximately >60-70% moisture can be converted to a biomass slurry extract.

Sugars and ethanol were measured in the biomass slurry extract using refractive index HPLC as performed by Midwest Laboratories Inc. Omaha Nebr. Fermentations containing yeast unexpectedly produced approximately 1.9% ethanol at a rapid rate of 12 hours from chopped corn biomass (See Table 3). Samples not treated with yeast contained only small amounts of ethanol detected at approximately 0.18% after 48 hours of fermentation. Glucose and fructose were non-detectable in yeast treated samples as early as 12 hours after initiation of fermentation possibly due to the rapid conversion to ethanol. It was also noticed that liquids were much easier to extract from yeast inoculated biomass following fermentation.

TABLE 3
Ethanol and Sugar Analysis of Liquid Samples
Following 48 Hours of Fermentation

| Sample | Glucose | Fructose | Ethanol |
|---|---|---|---|
| With yeast UB248L | n.d. | n.d. | 2.54% |
| With yeast UB236L | n.d. | n.d. | 2.65% |
| With yeast UB224L | n.d. | n.d. | 2.49% |
| With yeast UB212L | n.d. | n.d. | 1.89% |
| No yeast UA148L | 2.80% | 1.00% | 0.18% |
| No yeast UA124L | 2.60% | 1.10% | 0.11% |
| No yeast not fermented UA200L | 3.90% | 1.60% | 0.01% |

n.d. means not detected

Acid levels were measured in the biomass slurry extract specifically for lactic, acetic and butyric acid using refractive index HPLC as performed by Midwest Laboratories (Omaha, Nebr.). Surprisingly, inoculation of corn biomass with yeast had significant effects on the production of lactic acid producing 0.25% lactic acid on average after 48 hours of fermentation. Samples not treated with yeast produced approximately 9 fold more lactic acid (See Table 4). Lactic acid production was expected in the samples not treated with yeast since endogenous microbes are known to be present and able to produce lactic acid which is both beneficial and customary for silage production as livestock feed. It was also unexpectedly found that fermented yeast inoculated samples increase the production of acetic acid but reduced butyric acid by 40%. As shown in Table 4, yeast inoculation of fermentation samples also resulted in a higher pH value averaging approximately pH 5.24 over 48 hours of fermentation compared to pH 3.87 for samples without yeast inoculation. A more basic pH may offer, in some embodiments, more suitable conditions for enzymatic activity, higher ethanol yield, higher sugar yields, inoculation of microbes, easier transportation of products, less need for addition of chemicals to control pH and perhaps less maintenance costs. In some embodiments, acetic and butyric acid may be valuable by-products of the fermentation that can be sold or used in downstream applications (i.e. preparation of butonoate esters, preparation of butanol fuel, vinyl production, use of acetic acid as a chemical reagent to create other chemicals of commercial interest).

TABLE 4
Effects of Yeast Inoculation on Acid
Production and pH of Liquid Portion

| | Lactic | Acetic | Butyric | |
|---|---|---|---|---|
| Sample | Acid | Acid | Acid | pH |
| No Fermentation & No Yeast | 2.00% | 0.50% | 1.20% | 4.30 |
| Fermented No Yeast (24/48 hr. ave.) | 2.45% | 0.40% | 1.25% | 3.87 |
| Yeast Fermented (12/24/36/48 hr. | 0.25% | 0.73% | 0.75% | 5.24 |
| ave.) | | | | |

### Example 2

### Production of Plant Material for Examples 3-13

Greenhouse grown plant biomass was generated from two varieties of maize which were grown in irrigated pots. These plants were seeded in 4″ pots in December 2009 and allowed to germinate and grow for a period of approximately 2 weeks. The plants were then transplanted to 2 gallon pots and plant biomass harvested in February-April 2010.

Field grown plant biomass was generated at a % acre plot located near the town of Mebane in northern Orange County, N.C. The soil at the field site was fast draining sandy soil with very low organic matter and nitrogen content. The plot was managed by Eurofins. Hybrid corn was planted in a ⅛ acre of the field on May 28, 2010 using standard planting techniques. The seeds were spaced between 6 and 8 inches apart in 30″ rows. There were 10 rows of plants averaging 220′ long.

Forage sorghum variety FS-350 was planted in a 1/16 acre of the field in Mebane, N.C. on May 28, 2010 using standard planting techniques. The seeds were spaced between 1 and 1.5 inches apart in 30″ rows. There were 5 rows of plants averaging 220′ long.

Forage sorghum variety Graze n Bale was planted in a 1/16 acre of the field in Mebane, N.C. in May 2010 using standard planting techniques. The seeds were spaced between 1 and 1.5 inches apart in 30″ rows. There were 5 rows of plants averaging 220′ long.

Fertilization was managed by the application of 500 lbs per acre of a balance fertilizer blend (10-10-10) at the planting date, May 28, 2010, and an additional application of calcium ammonium nitrate (27-0-0) at a rate of 300 lbs per acre on Jun. 18, 2010. At planting a pre-emergent selective herbicide was used, Dual II Magnum, to control weeds at a rate of 16 oz per acre. One Jul. 27, 2010 a spot sprayer was used to apply Warrior insecticide to control corn ear worms at a rate of 3 oz per acre.

In addition to natural rainfall supplemental water was supplied via sprinklers periodically as need to maintain adequate soil moisture to keep plants from experiencing damaging drought conditions.

### Example 3

### Standard Methods of Analysis

Harvesting of Plant Material:

Maize and sorghum plants were harvested (referred to as plant biomass) from either the greenhouse or field by hand using pruners. The plants were cut at the base of the stalk near the soil level. After cutting the stalk the various tissues, including leaves, grain, seed and flower parts were removed from the stalks and sorted.

Processing of Plant Material:

Plant biomass was reduced to an average particle size of no more than ½ inch for all analyses by one of two methods. Less than 500 g of plant biomass was introduced into a standard type kitchen food processor equipped with multipurpose blades (Cusinart FP12). The instrument was turned on and the stalks and or leaves were fed in from the top of the device. The processor was run for 1-2 minutes to allow the plant material to be chopped finely. It was necessary to periodically stop the food processor and use a plastic spatula to mix the plant tissues to ensure even particle size reduction.

Larger amounts of plant material, greater than 1 kg was processed with a gasoline engine powered chipper shredder device for processing yard debris (Craftsman 8.5 Hp Chipper Shredder 77588 from Sears Roebuck and Company). The device produced an average particle size between ¼ and ½ inch.

Both of these methods produce a processed plant biomass similar in size and consistency to that produced by a commercial type silage chopper which would be the preferred device for commercial scale production of biomass slurry and biomass slurry extract.

Measuring Biomass and Moisture Content:

Biomass was measured by harvesting plant material and determining the mass and moisture content using precision balances and infrared moisture meters. The mass of the plant material was determined from either whole tissues or those that had been processed with either a food processor or chipper shredder. The moisture content of the plant material was determined only after processing had occurred using either a food processor or chipper shredder. The balance used for mass determination was the Denver Instruments Model S1-8002 and was operated according to the manufacturer's instructions. The moisture meter used to determine the moisture content of plant biomass was the Ohaus Model MB-45 operated according the manufacturer's instructions.

To determine the moisture content of plant biomass, approximately 8-10 gram sample of processed plant biomass was placed on the Ohaus MB-45 aluminum weigh pan and distributed evenly. The sample was dried at 100° C. using the standard one temperature program until the change in mass was less than 1 mg over 60 seconds. This method was confirmed several times by comparing the measurement against 100 gram samples dried in a vacuum oven at 95° C. for 24 and 48 hours.

Determining Sugar Content:

The sugar content of maize or sorghum plant biomass was determined by either a refractometer or an HPLC equipped with an analytical column suited for carbohydrate analysis and a refractive index detector. The plant biomass was harvested using hand pruners and processed into small pieces as described above. This processed biomass was then introduced into a modified French press to squeeze the juice from the solid tissue by the method described below. This extracted juice was analyzed directly for sugar content using a refractometer (Extech Instruments model RF15 Portable Sucrose Brix Refractometer ((0 to 32%) with ATC).

To measure the sugar content using a refractometer, a small volume of the juice, approximately 0.5 ml, was placed on the glass prism of a hand held refractometer equipped to measure Brix from 0-32°±0.2° which included automatic temperature compensation. The plastic cover slip was lowered onto the sample and the value of sugar content was determined by viewing through the objective lens and identifying where the blue bar ends and comparing that to the Brix scale located inside the viewfinder. The refractometer was cleaned with distilled water and tissue paper between samples.

To measure the sugar content and composition an HPLC was used.

Pressing Plant Tissues to Extract Juice:

Liquid was extracted from approximately 20 grams of plant biomass using a French Press (model FA-078A from Thermo Electron) following manufacturers instructions; however, the instrument was modified to accommodate the collection of crude liquid extract derived from the application of high pressure to chopped and shredded plant tissues. This crude extract contains suspended solids of such a size that prevent flow of liquid from the small diameter collection tube installed by the manufacturer. The modification involved the removal of the small diameter collection tube and replacing it with one that had approximately ⅛″ inside diameter. The use of the larger drain tube prevented solid particles from accumulating inside this tube. A supplemental filter aid comprised of cheese cloth was added to the piston before addition of the sample itself to reduce flow problems.

The rubber o-rings for the cylinder plug and piston were lubricated with silicon grease between each sample. The plug was inserted into the bottom of the cylinder. Into the cylinder approximately 5 grams of cheesecloth was inserted and pressed to the bottom. The sample was then added and gently packed down. Finally the piston was carefully placed into the cylinder. This assembly was then placed into the French press apparatus and 500 psi was applied to the piston. The extracted juice flowed from the collection spout located on the side of the bottom of the cylinder. Liquid exudates were collected in a 50 ml plastic tube.

Purification of Liquid Samples for HPLC Analysis:

Sedimentation and filtration was used to purify samples prior to HPLC analysis. For sedimentation, a portion of the extracted juice was introduced into a 1.8 ml microcentrifuge tube and spun at 15,000 rpm for 5 minutes. After the spin, the clarified supernatant was transferred to another microcentrifuge tube equipped with a 0.45 μm membrane filter. The tube and sample was then spun at 5,000 rpm for 5 minutes. The liquid sample was collected from the bottom of the filtration tube. For samples that were measured as having a Brix value >10, a dilution of the sample (with distilled and dionized water) may have been required prior to analysis.

Assembling Fermentation Reactions:

To assemble the fermentation reactions, the plant biomass was harvested and physically processed as described above under the heading “Processing of plant material”. The amount of time between the harvest and the use of plant material was minimized by processing the plants immediately after harvest and setting up fermentation reactions within one hour. A large amount of plant biomass was used for the harvest and processing to ensure a representative sample of plant biomass was used for all of the experimental conditions within each experiment. For example, if 10 reactions were assembled, each using a mass of 100 grams of plant biomass, 2 or more kilograms of plant biomass was harvested and processed. The processed plant biomass was added to a large sanitized plastic container to increase the surface area of the biomass.

Additives such as yeast inoculants or liquid enzymes were typically only a small fraction of the overall mass of the fermentation reaction. To ensure the fermentation reaction was mixed well, the additives were dispersed evenly over the surface of the freshly harvested and processed plant biomass. Following the addition of any additives, the processed plant biomass was hand tossed and turned over and over for a few minutes. For reactions greater than 1 kilogram, a fraction of the total amount of plant biomass required was mixed with the appropriate fraction of any necessary additives and once incorporated, the fractions were pooled to generate the total mass of such a fermentation reaction. For industrial scale application of the fermentation process, the silage chopper would be coupled with a device capable of simultaneous inoculating with any microbes and supplying continuously any other additives needed.

Reaction Vessel Type, Preparation and Incubation:

The fermentation process which produces biomass slurry and biomass slurry extract was designed to take place in a silo in which the harvested, processed and inoculated plant biomass is packed to eliminate pockets of air. This leads to an anaerobic environment which favors the types of fermentation necessary to produce the products of alcohols and organic acids. To simulate the use of a silo in which little to no air is present as a result of the tight packing of plant biomass, two strategies were employed for these lab scale evaluations.

The first type of vessels used to contain fermentation reactions were new plastic vacuum seal bags. These bags were used to conduct fermentations at a small scale using approximately 100 grams of plant material. The plant material was introduced into a new plastic bag and moved towards the bottom of the pouch. The bag was then placed into the vacuum sealer device and a vacuum and seal were created following the manufacturer's instructions. The bags and the device used to create the vacuum are manufactured by the Food Saver Company. The model of the vacuum sealer is V2450 Vacuum Sealer and the bag type used is model FSFSBF0116, FSFSBF0226, and FSFSBF0326 Pint, Quart and Gallon Sized Bags.

The second type of vessel was employed when larger amounts of biomass were used. This vessel was a 5 gallon plastic bucket with a tight sealing lid or 100 L water tank with a custom tight fitting insulating top designed to sit just atop the substrate. With either of these two vessels, the container was first cleaned with a lab grade cleanser, rinsed with tap water and then sanitized using an iodine-based, no-rinse sanitizer. The plant biomass was introduced into the reaction vessels in layers that were approximately ⅕ of the total mass used for each reaction size. After adding each layer the biomass was packed tightly to eliminate any air pockets. At the same time the 3^{rd} layer of biomass was being added to the fermentation vessel a small thermocouple wire was inserted into the center of the reaction matrix. This thermocouple was then connected to a temperature probe to monitor the temperature of the reaction. Once all of the plant biomass was added, the vessel was sealed with a tight fitting cover or lid. Because the process involves the production of gases during fermentation, some small vents were created to allow the pressure to dissipate.

Yeast Dosing:

The use rate of yeast (Ethanol Red from Fermentis) in these studies was very high, 4 grams of yeast per 100 grams of plant biomass. This allows for maximum conversion of sugars into alcohol by yeast while minimizing the development of additional yeast biomass. To use the dried yeast, the appropriate amount of dried yeast was weighed out using a precision balance. The dried yeast was then sprinkled over top of freshly harvested and processed plant biomass and mixed well.

Enzyme Usage:

Two commercially available enzyme products were used during of the fermentation process. Both of these enzymes were formulated as viscous liquids. The first enzyme was Accellerase 1000 (Genencor) and the second enzyme was Celluclast (Sigma Chemical Company). To use the liquid enzymes the appropriate amount of enzyme was drawn into a pipet and dispensed drop wise and evenly over the surface of freshly harvested and processed plant biomass.

Temperature Monitoring:

The nature of the biomass fermentation process, namely a solid state fermentation, makes fine temperature control of the process difficult. It is probable that the reaction temperature would be that which occurs naturally when industrially relevant amounts of plant biomass are reacted using the fermentation process. The temperature of the fermentation reactions was monitored using a digital data logging thermometer and thermocouple. The data logger was the Sper Scientific Four Channel model #800024. This instrument was connected to 4 individual thermocouples model #800077. The devices were operated according to the manufacturer's instructions and the data logger was set to capture a temperature measurement every 15 minutes from each thermocouple. The stored information was downloaded and processed from the instrument using the manufacturer's software and then loaded into standard plotting programs for additional presentation and analysis.

HPLC Analysis of Liquids for Sugars, Alcohols and Organic Acids:

HPLC analytical methods were used and developed to measure the type and concentration of sugars, alcohols and organic acids present in the plant biomass and fermentation. The instrument used for these analyses was a Waters Breeze equipped with a refractive index detector and one of two types of columns from Biorad, the HPX-87C and HPX-87H. The manufacturer's operating instructions were followed for both the instrument and the analytical columns.

The HPX-87C column was used to measure the amount of glucose, fructose and sucrose present in liquid extracts obtained from plant tissues. This column was operated at 85° C. using water as the mobile phase at a flow rate of 0.6 ml/min with the refractive index detector set to a temperature of 40° C. The injection volume for samples and standards was 25 μl.

The HPLX-87H column was used to measure the amount of glucose, fructose, lactic acid, acetic acid, glycerol and ethanol obtained from plant tissues before, during and after the application of the fermentation process. This column was operated at 65° C. using 0.005M Sulfuric Acid as a mobile phase at a flow rate of 0.6 ml/min. The refractive index detector was set to a temperature of 45° C. The injection volume for samples and standards was 25 μl.

Each instrument was calibrated with a set of 5 standards. These standards were prepared by dissolving or mixing known amounts of each component in water to create a solution of a specific concentration. This solution was diluted volumetrically to create 5 unique calibrants of various concentrations. One calibrant was created for use with the HPX87-C column containing glucose, fructose and sucrose. Another calibrant was created for use with the HPX87-H column which contained glucose, maltose, acetic acid, lactic acid, glycerol and ethanol.

The standards were used to create a calibration curve for each component to be quantified using linear regression. From these curves, the value for each component in a sample was determined and is represented as % w/v present in that sample. Corrections for dilution were applied as needed.

### Example 4

### Stem Tissue is the Preferred Substrate for the Generation of Biomass Slurry and Biomass Slurry Extract

To determine which plant tissue was preferred for generating biomass slurry and biomass slurry extract, several plant tissues were tested for the production of ethanol and organic acids over a period of time. The amount of sugar and moisture in the harvested plant biomass was determined using a refractometer, an HPLC equipped with the BioRad HPX87-H column and both a vacuum oven and an infrared moisture meter essentially as described in Example 3.

The plants taken from the greenhouse were early in the reproductive cycle and therefore the majority of the tissues from the aerial portions of the plants were either leaf or stem. These maize plants were harvested early in 2010 at a stage when the ears were in an early stage of development and tassels were still shedding pollen. The various aerial portions of the plant were divided into stems, leaves and reproductive tissues. At this early stage of development the reproductive tissues represented a minor portion of the total biomass and were not included in any additional investigations. These two major tissue types were processed using a food processor. Some of the processed tissue was used to determine the moisture content of each tissue type. The vegetative tissues were comprised of approximately ⅔ stem tissue and ⅓ leaf tissue on either a dry or fresh basis.

Juice was pressed from a portion of the processed tissues for analyses using both the hand held refractometer for Brix and the HPLC essentially as described in Example 3. Table 5 describes the moisture content of the harvested tissue using two different methods. Table 6 describes the sugars, alcohols and organic acids present in the harvested tissue prior to any further fermentation.

TABLE 5
Moisture content of harvested greenhouse tissue.

| | % moisture | % moisture | |
|---|---|---|---|
| tissue | measured by | measured by | |
| type | IR meter | vacuum oven | Brix |
| leaf | 77.5 | 76.3 | 11 |
| stalk | 77.9 | 75.6 | 14 |

TABLE 6
Sugar, alcohol and organic acid content of harvested greenhouse tissue.

| | | Total mono | | | | |
|---|---|---|---|---|---|---|
| | | and | Lactic | | Acetic | |
| | | disaccaride | Acid | Glycerol | Acid | Ethanol |
| Tissue Type | Time Point | sugars % w/v | % v/v | w/v % | v/v % | w/v % |
| stalk | pre-fermentation | 9.0 | 0.0 | 0.0 | 0.0 | 0.0 |
| leaf | pre-fermentation | 3.7 | 0.0 | 0.1 | 0.2 | 0.0 |

The amount of sugar as measured by the refractometer and the HPLC do not correspond exactly. It is known that other soluble compounds and various types of sugars affect the Brix value obtained from the refractometer. Based on the more accurate HPLC analysis it is clear that the Brix value should be considered as an estimate of the actual sugar concentration in these samples. The two types of sugar measurements indicate that the leaf samples contained less sugars when compared to the stem or stalk tissue. This data also shows that the amount of moisture present in each of the tissues was very similar and the two methods for moisture determination were very similar. Considering both the moisture content and the relative concentration of sugar in each tissue type, the stem or stalk tissues are likely to contain more sugars for fermentation. Future moisture measurements were made exclusively with the infrared moisture meter.

The remaining tissues harvested and processed from greenhouse grown maize were used to conduct fermentation reactions to generate biomass slurry and biomass slurry extract. Three types of samples, 100 g for each type, were fermented using only dried yeast and no other additives. The first type of sample fermented was a 1:1 mixture by mass of stem and leaf tissues, the second type was leaf only and the third type was stem or stalk only. Fermentation was performed essentially as described in Example 3, after mixing the reaction components the ferment was sealed in vacuum bags. The vacuum bags were incubated at 30° C. for 72 hours. At 3 time points, 24, 48 and 72 hours after initiation of the ferment, the vacuum bags were opened and approximately 5 g of biomass slurry was removed for HPLC analysis. The bags were resealed after the sample was taken. The results of the HPLC analysis are shown in Table 7.

TABLE 7
Composition of biomass slurry extract of greenhouse grown biomass over time.

| | | Total | | | | |
|---|---|---|---|---|---|---|
| | | mono and | | | | |
| | | disaccaride | Lactic | | Acetic | |
| | | sugars | Acid | Glycerol | Acid | Ethanol |
| Tissue Type | Time Point | % w/v | % v/v | w/v % | v/v % | w/v % |
| leaf stem mix | 24 hrs post-fermentation start | 0.3 | 0.5 | 0.5 | 0.3 | 2.9 |
| leaf | 24 hrs post-fermentation start | 0.3 | 0.6 | 0.4 | 0.3 | 1.7 |
| stem | 24 hrs post-fermentation start | 0.4 | 0.5 | 0.5 | 0.2 | 4.2 |
| leaf stem mix | 48 hrs post-fermentation start | 0.3 | 0.4 | 0.5 | 0.5 | 2.9 |
| leaf | 48 hrs post-fermentation start | 0.3 | 0.7 | 0.2 | 0.4 | 1.6 |
| stem | 48 hrs post-fermentation start | 0.3 | 0.6 | 0.5 | 0.3 | 4.1 |
| leaf stem mix | 72 hrs post-fermentation start | 0.1 | 0.4 | 0.4 | 0.7 | 2.9 |
| leaf | 72 hrs post-fermentation start | 0.0 | 0.1 | 0.1 | 0.7 | 1.4 |
| stem | 72 hrs post-fermentation start | 0.1 | 0.8 | 0.5 | 0.4 | 4.2 |

This data demonstrates that different tissues when processed to produce biomass slurry and biomass slurry extract produce different concentrations of the products ethanol, lactic acid, glycerol and acetic acid. The sum of these four products can be used to estimate the relative potential of each tissue as a substrate. Stem based biomass produced the highest concentrations of each of these 4 products in almost each case and therefore the stem is a preferred tissue type. The soluble simple sugars were converted to ethanol rapidly, within 24 hours. The development of greater concentrations of lactic and acetic acids as well as glycerol during processing suggest that another microbe or enzyme was hydrolyzing carbohydrates within the plant tissues up to the 72 hour time point.

Another maize plant also grown in a greenhouse was harvested and separated into the leaf and stem tissues. These tissues were processed and the juice was extracted essentially as described in Example 3. The juice was filtered and analyzed on an HPLC with the HPX87-C column to understand the composition of the sugars within the juice and to specifically measure sucrose, glucose and fructose which the HPX87-H cannot. Analysis of sugar content for pre-fermented samples was performed using the HPX87-C column and analysis of samples after fermentation had been initiated were performed on the HPX87-H. The results for this analysis are shown in Table 8.

TABLE 8
Composition of greenhouse grown plant
material using HPX87-H column.

| tissue | Sucrose | Glucose | Fructose | Total sugar |
|---|---|---|---|---|
| type | % w/v | % w/v | % w/v | % w/v |
| leaf | 3.9 | 1.4 | 0.7 | 6.1 |
| stalk | 5.7 | 1.6 | 0.8 | 8.2 |

As with the previously analyzed samples of maize leaf and stem tissues the stem tissue in this evaluation contained a greater concentration of sugars compared to leaf tissue. This suggests that the stem tissue is a more desirable substrate for the generation of biomass slurry and biomass slurry extract.

### Example 5

### Fermentation of Field Grown Plant Biomass

Plants grown in Mebane, N.C. were sampled several times over a 3 month period to monitor the development of various plant tissues and the relative composition of the total biomass produced in the field. The plants monitored were maize and two varieties of forage sorghum, Graze n' Bale and FS350 grown essentially as described in Example 2. To achieve a representative sample, a 6′ row of each was harvested at each of the time points. Plants were harvested by hand using pruners and divided into 3 different tissue types, leaves, stalks or stems and reproductive tissues which would include inflorescences, grain and any supportive tissues. These portions were then measured for mass using a precision balance essentially as described in Example 3. The data for these measurements is shown in Table 9.

TABLE 9
Moisture content of field grown plant biomass over time.

| Plant Type | Tissue Type | Aug. 2, 2010 | Aug. 24, 2010 | Aug. 31, 2010 | Sep. 9, 2010 | Sep. 21, 2010 | Oct. 7, 2010 | Nov. 9, 2010 |
|---|---|---|---|---|---|---|---|---|
| Maize | stalks | 49% | 46% | 46% | 46% | 46% | n/a | n/a |
| Graze n' Bale | stalks | n/a | 73% | 73% | 75% | 75% | 74% | 74% |
| FS350 | stalks | n/a | 82% | 82% | 83% | 83% | 80% | 80% |
| Maize | leaves | 20% | 13% | 13% | 9% | 9% | n/a | n/a |
| Graze n' Bale | leaves | n/a | 27% | 27% | 25% | 25% | 26% | 26% |
| FS350 | leaves | n/a | 14% | 14% | 12% | 12% | 7% | 7% |
| Maize | reproductive | 32% | 41% | 41% | 45% | 45% | n/a | n/a |
| | tissues | | | | | | | |
| FS350 | reproductive | n/a | 4% | 4% | 5% | 5% | 13% | 14% |
| | tissues | | | | | | | |
| Graze n' Bale | reproductive | n/a | n/a | n/a | n/a | n/a | n/a | n/a |
| | tissues | | | | | | | |

The stalks represent varying percentages of harvestable material from field grown corn and sorghum plants. As demonstrated in Example 4, stalks were very efficacious plant biomass for use in generating biomass slurry and biomass slurry extract. While any plant biomass can be used, plants that generate more stalks than leaves, ears or flowers are preferable. Furthermore the stalks may represent the majority of plant biomass available if harvested later in the growing season.

An analysis was performed to determine the relationship between the Brix values obtained from the refractometer and sugar content as measured by an HPLC equipped with a HPX-87C column. Using field grown forage sorghum, variety FS350, 50 individual plants were harvested. One large cane from each of the plants was processed and the liquid extract was measured for sugar content using both the refractometer and the HPLC. The Total Sugars % w/v data in Table 10 represents the sum of the concentrations of sucrose, glucose and fructose. The above was performed essentially as described in Example 3 and the results from this analysis are shown in Table 10.

TABLE 10
Sugar analysis of field grown sorghum.

| Forage Sorhum plant # | Brix | Total Sugars % w/v |
|---|---|---|
| 1 | 8.8 | 7.0 |
| 2 | 9 | 8.2 |
| 3 | 9 | 7.8 |
| 4 | 9.2 | 6.9 |
| 5 | 9.6 | 8.1 |
| 6 | 9.8 | 8.8 |
| 7 | 10.2 | 9.9 |
| 8 | 10.4 | 9.1 |
| 9 | 10.6 | 9.4 |
| 10 | 10.6 | 9.8 |
| 11 | 10.6 | 9.5 |
| 12 | 10.8 | 10.7 |
| 13 | 10.8 | 9.9 |
| 14 | 10.8 | 9.7 |
| 15 | 11 | 11.2 |
| 16 | 11 | 10.5 |
| 17 | 11 | 10.7 |
| 18 | 11.2 | 11.1 |
| 19 | 11.2 | 11.6 |
| 20 | 11.2 | 11.4 |
| 21 | 11.2 | 10.6 |
| 22 | 11.4 | n/a |
| 23 | 11.6 | 10.7 |
| 24 | 11.6 | 10.1 |
| 25 | 11.8 | 11.6 |
| 26 | 12 | 12.1 |
| 27 | 12 | 12.3 |
| 28 | 12 | 12.2 |
| 29 | 12 | 11.9 |
| 30 | 12 | 9.9 |
| 31 | 12 | 12.2 |
| 32 | 12 | 12.1 |
| 33 | 12.2 | 12.6 |
| 34 | 12.2 | 12.5 |
| 35 | 12.2 | 13.0 |
| 36 | 12.2 | 11.6 |
| 37 | 12.2 | 12.2 |
| 38 | 12.3 | 12.7 |
| 39 | 12.6 | 12.5 |
| 40 | 12.8 | 13.6 |
| 41 | 12.8 | 13.1 |
| 42 | 12.8 | 12.9 |
| 43 | 12.8 | 12.7 |
| 44 | 13 | 13.6 |
| 45 | 13 | 13.5 |
| 46 | 13 | 13.3 |
| 47 | 13.4 | 13.2 |
| 48 | 13.4 | 13.0 |
| 49 | 13.4 | 13.3 |
| 50 | 14.2 | 14.1 |

The data from each method (refractometer versus HPCL) correlate extremely well with one another and it is therefore reasonable to consider the use of a hand held refractometer for field use when monitoring the development of tissues with respect to sugar content. The Brix value is an estimate of the amount of sugar present; however, it does not give an indication of the composition of the sugar measured. The use of an HPLC for measuring sugar content and composition was the most reliable and accurate means to understand both the concentration and type of sugars in liquid extracted from plant biomass. The HPLC analysis required much more sample preparation, took more than 30 minutes to perform and required access to expensive equipment. The use of a refractometer was a preferred method to indicate in a general way the potential of a given plant biomass to produce biomass slurry and biomass slurry extract. While the refractometer was not able to distinguish between various sugars and was affected by the presence of other soluble compounds in the plant's juices, it was very fast and the instrument was inexpensive. The data in Table 10 also suggest there was some variation in the sugar content of juice extracted from plants of the same variety grown next to one another. This data indicated that comprehensive assessments of productivity of plant biomass would best be managed by study of large amounts of plant material (such as greater than a single plant).

Forage Sorghum plants were harvested and analyzed using the HPLC equipped with the HPX87-C column essentially as described in Example 3. The stalks were pressed and the juice extracted from this biomass was subjected to sugar analysis to determine the composition of the sugars in the juice. Table 11 contains the sugar analysis of the harvested sorghum plants. The sugars present in field grown sorghum plants were a mix of sucrose, glucose and fructose and that the ratio of these sugars changed as the plants matured during the growing season. The concentration of both glucose and fructose remain nearly constant and the concentration of sucrose increased significantly during plant development. This data also demonstrates that the overall amount of sugars contained within sorghum increases as the plant matures.

TABLE 11
Sugar analysis of field grown forage sorghum.

| | | | | | Total | |
|---|---|---|---|---|---|---|
| Sample | | Sucrose | Glucose | Fructose | Sugar | |
| Name | Date | % w/v | % w/v | % w/v | % w/v | Brix |
| FS350 | Sep. 21, 2010 | 5.1 | 4.3 | 2.6 | 12.1 | 14 |
| FS350 | Sep. 29, 2010 | 7.1 | 4.6 | 2.8 | 14.4 | 14 |
| FS350 | Oct. 6, 2010 | 8.8 | 3.7 | 2.7 | 15.2 | 15 |
| FS350 | Nov. 9, 2010 | 16.2 | 4.4 | 2.9 | 23.5 | 18 |
| Graze | Sep. 21, 2010 | 2.1 | 4.6 | 2.1 | 8.8 | 10 |
| n' Bale | | | | | | |
| Graze | Sep. 29, 2010 | 4.6 | 3.0 | 1.8 | 9.4 | 10 |
| n' Bale | | | | | | |
| Graze | Oct. 6, 2010 | 4.4 | 4.4 | 2.0 | 10.9 | 12 |
| n' Bale | | | | | | |
| Graze | Nov. 9, 2010 | 9.4 | 3.9 | 2.2 | 15.4 | 14 |
| n' Bale | | | | | | |

### Example 6

### Time from Harvest to Start of the Fermentation Process

A study was conducted to determine what affect the delay of chopping or inoculation of yeast would have on the composition of biomass slurry or biomass slurry extract using only yeast and those microbes which exist naturally on the plants at the time of harvest. Maize was field grown in Mebane, N.C. essentially as described in Example 2. Approximately 20 kg of plant biomass was harvested and any reproductive structures were discarded and the remaining stalks were cut to approximately 2′ sections using hand pruners. Seven different 1 kg reactions were assembled using various temporal delays in either the chopping of the plant biomass or the inoculation with yeast. Each reaction was contained within 1 gallon vacuum seal bags and received 40 g dried yeast essentially as described in Example 3.

The control reaction was produced from plant biomass that was harvested, chopped and inoculated immediately after harvest. In addition to the control reaction, approximately 5 kg of maize stalks were chopped just after harvest and reserved in 1 kg portions inside 1 gallon vacuum seal bags. This reserved, un-inoculated plant material served as the substrate for three reactions that were harvested and chopped but received an inoculation at various hours after chopping. The delays were 4, 8 and 24 hours respectively. Another 5 kg portion of maize stalks was reserved as un-chopped material and stored in a large plastic bag inside a cooler kept at ambient temperature, approximately 30° C. At 4, 8 and 24 hours respectively, a 1 kg sample of this un-chopped maize tissue was chopped and inoculated with 40 g dried yeast and then added to a 1 gallon vacuum seal bag.

All of the stalks of maize plants for this study were chopped and processed essentially as described in Example 3. All 1 kg reactions received a 40 g inoculum of Ethanol Red dried yeast. The plant material for this experiment was processed using the food processor. The biomass and yeast were well mixed by hand in a small tub and then placed inside a 1 gallon vacuum seal bag and sealed. All reactions were allowed to proceed for approximately 72 hours in an incubator set to 30° C. All reactions were sampled at approximately 72 hours after initiation of the ferment, the vacuum bags were opened and a small sample, approximately 5 g was removed for HPLC analysis using the BioRad HPX87-H column. A summary of the reaction details and the results of the HPLC analysis are show in Table 12.

TABLE 12
Analysis of ferment while varying time to start the process

| | Chopping | Inoculum | Total | |
|---|---|---|---|---|
| | time after | time after | Organic | Ethanol |
| Reaction | harvest (hrs) | harvest (hrs) | Acids % v/v | w/v % |
| 1 kg Maize Stems | 0 | 0 | 1.3 | 4.2 |
| 1 kg Maize Stems | 0 | 4 | 2.5 | 3.8 |
| 1 kg Maize Stems | 4 | 4 | 0.5 | 3.6 |
| 1 kg Maize Stems | 0 | 8 | 2.8 | 3.6 |
| 1 kg Maize Stems | 8 | 8 | 0.8 | 2.6 |
| 1 kg Maize Stems | 0 | 24 | 3.3 | 2.9 |
| 1 kg Maize Stems | 24 | 24 | 3.1 | 2.9 |

This data demonstrates that a temporal delay between harvest, chopping and inoculation with yeast will lead to the development of different concentrations of organic acids and ethanol. Any delay in the inoculation of plant biomass which has been harvested and chopped will lead to an increase in the production of organic acids and a decrease in the final concentration of ethanol. To maximize the production of ethanol, the timing of harvest, chopping and inoculation was very important and should be completed in as little time as possible; however, this time can vary if the desired output is organic acids.

For samples which were chopped and inoculated following a delay after harvesting, the organic acid and ethanol concentration after fermentation was in most cases less than either the control reaction or those samples which were harvested and chopped immediately experiencing only a delay in inoculation with yeast. Samples that experienced a delay in inoculation after harvesting and chopping showed an increase in the production of organic acids and a reduced accumulation of ethanol following fermentation compared to either the control or those samples that experienced a delay in both chopping and inoculation. While not intending to limited by theory, it is likely that naturally occurring fungi and bacteria begin consumption of sugars and fibers at an increased rate when not competing with a large inoculum of yeast. This is true mainly for those plant tissues that have been chopped immediately after harvest. The increase in organic acids and decrease in ethanol are consistent with this observation. The samples which were harvested and not chopped appear to have a decrease in both organic acids and ethanol which suggests that the plant tissues themselves reduce the availability of compounds utilized during the fermentation process.

### Example 7

### Addition of Cellulolytic Enzymes at 100 g of Biomass Scale

Plant biomass was grown, harvested and processed essentially as described in Examples 2 and 3. The plant biomass was stems or stalks from maize grown in the greenhouse and processed using a food processor. Plant biomass was inoculated with dried yeast with or without the addition of the liquid enzyme product Accellerase 1000 (Genencor). Approximately 250 g of chopped plant biomass was combined with 10 g of Ethanol Red yeast. This material was mixed well and two 100 g aliquots were made. Into one aliquot 1.3 ml of liquid enzyme Accellerase 1000 was added and the second aliquot received 1.3 ml of distilled water and 4 g additional of Ethanol Red yeast. Once the liquid addition was made the plant material was mixed again by hand. The material was added to a 1 pint vacuum bag and sealed. The fermentation was allowed to proceed in a 30° C. incubator for a period of 72 hours. At 3 time points, 24, 48 and 72 hours after initiation of the ferment, the vacuum bags were opened and a sample of the biomass slurry, approximately 5 g, was removed for HPLC analysis. The bags were resealed after the sample was taken. The results of the HPLC analysis are show in Table 13.

TABLE 13
HPLC analysis of biomass slurry with and without the addition of enzymes.

| | | Inoculated | | | | |
|---|---|---|---|---|---|---|
| | | Biomass and | | Inoculated | | Inoculated |
| | Inoculated | Accellerase | Inoculated | Biomass and | Inoculated | Biomass and |
| | Biomass | 1000 | Biomass | Accellerase | Biomass | Accellerase |
| Time Point | (Ethanol | (Ethanol | (Lactic Acid | (Lactic Acid | (Acetic Acid | (Acetic Acid |
| (hours) | w/v %) | w/v %) | w/v %) | w/v %) | w/v %) | w/v %) |
| 24 | 2.2 | 2.8 | 0.4 | 0.5 | 0.1 | 0.2 |
| 48 | 2.2 | 2.7 | 0.5 | 0.8 | 0.2 | 0.4 |
| 72 | 2.0 | 2.7 | 0.5 | 1.2 | 0.3 | 0.5 |

Results of the HPLC analysis indicate that the addition of the cellulolytic enzyme Accellerase 1000 to the fermentation increased the production of ethanol compared to the control. In addition, ethanol production appears to be complete in approximately 24 hours independent of whether enzymes were added. The addition of cellulolytic enzymes also increased the production of organic acids which continued to increase even at 72 hours fermentation. While not intending to be limited by theory, the organic acids are likely accumulating due to the activity of bacteria which entered the fermentation process by being present in the plant biomass. These bacteria are likely producing lactic acid which is expected to be a large portion of the organic acid measured. The total concentration of organic acids produced in the reaction containing the added enzyme was more than twice that of the control.

The duration of the fermentation process can be altered depending upon the specific products desired in the biomass slurry. For instance, fermenting for 24 hours will obtain the majority of ethanol that will be produced; however, fermenting for more than 24 hours and perhaps in excess of 72 hours will result in the production of organic acids.

### Example 8

### Large Scale Fermentations

The plant biomass for this experiment was maize grown in the field and processed using the chipper shredder essentially as described in Examples 2 and 3. Approximately 13 kg of chopped plant biomass was combined with 520 g of Ethanol Red dried yeast and mixed by hand to form an inoculated biomass. The inoculated biomass was partitioned into three separate reaction sizes, 100 g, 1 kg and 10 kg.

Two 100 g aliquots of inoculated biomass were placed into individual quart sized bags and sealed with a vacuum sealer. One of these 100 g reactions was placed into a 32° C. incubator and the other was allowed to react at ambient conditions in a shaded area outside. Two 1 kg aliquots of inoculated biomass were placed into individual gallon sized bags and sealed with a vacuum sealer. One of these 1 kg reactions was placed into a 32° C. incubator and the other was allowed to react at ambient conditions in a shaded area outside. A 10 kg aliquot of inoculated biomass was added to a 5 gallon bucket. The bucket was sealed with a tight fitting lid. One of each of the 100 g and 1 kg reactions and the 10 kg reaction were allowed to react at ambient temperatures and no attempt was made to control the environment. The ambient conditions in Mebane, N.C. at this time of year were on average 28-32° C. The reactions were allowed to proceed for approximately 72 hours. Samples of the contents of each of the reactions were taken after 72 hours for analysis of ethanol content and organic acid content. Ethanol and organic acid content was determined through analysis using an HPLC equipped with the BioRad HPX87-H column. Table 13 outlines the data generated when comparing samples of varying starting biomass quantity with or without control over the reaction temperature.

TABLE 13
Composition of samples from varying the starting quantity of biomass
in the fermentation and varying the ambient temperature.

| | | | |
|---|---|---|---|
| | | Total | |
| | Temperature | Organic | Ethanol |
| Reaction Size | of fermentation | Acids % v/v | w/v % |

| | | | | |
|---|---|---|---|---|
| 100 | g | 32° C. | 0.6 | 4.0 |
| 100 | g | ambient | 0.4 | 3.8 |
| 1 | kg | 32° C. | 0.6 | 3.7 |
| 1 | kg | ambient | 0.5 | 3.7 |
| 10 | kg | ambient | 0.4 | 3.8 |

The data suggest that there are no major differences when processing varying quantities of biomass. The control of temperature also did not seem to have any significant impact on performance at the 100 g or 1 kg scale.

### Example 9

### 100 g Scale Fermentation with Cellulolytic Enzymes

Forage Sorghum, variety FS350, was grown, harvested and processed essentially as described in Examples 2 and 3. The plant material was processed using the chipper shredder. Approximately 65 kg of chopped plant biomass was combined with 2600 g Ethanol Red dried yeast to form inoculated biomass. The biomass and yeast were well mixed by hand in smaller batches of approximately 5 kg.

Inoculated biomass was partitioned to create 5 different 100 g reactions. Into the first reaction, which was considered the control, 5.2 ml of distilled water was added. The remaining four reactions each received a unique combination of one of the two enzymes and one of the two doses of these enzymes. For those two reactions employing the smaller dose of enzyme an addition of water was made to bring the total volume of all liquid added to the reaction to 5.2 ml. The liquid additions were balanced for all reactions to facilitate comparison of the reaction products ethanol and organic acids. The compositions of reactions at the time of assembly are shown in Table 14.

TABLE 14
Sorghum biomass and varying concentrations
of enzymes in the fermentation.

| | | Reaction | Enzyme | Water |
|---|---|---|---|---|
| Sample # | Enzyme type | mass (g) | added (ml) | added (ml) |
| 1 (Control) | n/a | 100 | 0.0 | 5.2 |
| 2 | Celluclast | 100 | 2.6 | 2.6 |
| 3 | Celluclast | 100 | 5.2 | 0.0 |
| 4 | Accellerase 1000 | 100 | 2.6 | 2.6 |
| 5 | Accellerase 1000 | 100 | 5.2 | 0.0 |

After adding the enzymes and water as needed, the reactions were well mixed by hand and the inoculated biomass was placed into a 1 quart vacuum seal bag and sealed. These reactions were placed into an incubator set to 32° C. for approximately 72 hours. Samples of the reactions were taken and ethanol and organic acid content was determined using an HPLC equipped with a BioRad HPX87-H. Table 15 outlines the data generated when comparing samples with and without the additions of the two doses of liquid enzyme.

TABLE 15
Composition of biomass slurry of sorghum and varying concentrations of enzyme.

| | | | | | | % of | % of organic |
|---|---|---|---|---|---|---|---|
| | | Enzyme Dose | | | | ethanol vs. | acids vs. no |
| | | (ml enzyme/g | Lactic Acid | Acetic Acid | Ethanol | no enzyme | enzyme |
| Reaction Size | Enzyme Type | biomass) | v/v % | v/v % | w/v % | control | control |
| 100 g | none | none | 0.4 | 0.6 | 3.6 | 100% | 100% |
| 100 g | Accellerase 1000 | 2.6 | 0.9 | 0.6 | 4.4 | 123% | 164% |
| 100 g | Accellerase 1000 | 5.2 | 1.1 | 0.7 | 4.5 | 127% | 197% |
| 100 g | Celluclast | 2.6 | 2.9 | 0.5 | 4.4 | 123% | 362% |
| 100 g | Celluclast | 5.2 | 2.7 | 0.5 | 4.4 | 124% | 339% |

The data shows that the addition of cellulolytic enzymes increases the amount of ethanol and organic acids produced.

### Example 10

### 50 kg Scale Fermentation with Cellulolytic Enzymes

Forage Sorghum, variety FS350, was grown, harvested and processed essentially as described Examples 2 and 3. The plant biomass for this experiment was grown in the field and processed using the chipper shredder. Approximately 65 kg of chopped plant biomass was combined with 2600 g Ethanol Red dried yeast. The biomass and yeast were well mixed by hand in smaller batches of approximately 5 kg. Inoculated biomass was divided into two portions, a 10 kg portion to which no enzyme was added and a 50 kg portion to which Celluclast (Genencor) enzyme was added at a rate of 2.6 ml per 100 g of biomass.

The 10 kg inoculated biomass was placed in small amounts into a 5 gallon bucket and packed tightly as it was being added. The 10 kg reaction contained no exogenous enzymes. Into the center of the inoculated biomass a thermocouple was inserted and chopped plant biomass was added to fill the container. The container was closed with a tight fitting lid.

The 50 kg portion of inoculated biomass was mixed with 1.3 L of Celluclast enzyme in small batches and added to a 100 L fermenter. A thermocouple was added to the center of the fermentation reaction to monitor the temperature. An insulated cover was placed directly on the top of the packed inoculated biomass to seal off the ferment from the atmosphere because the 100 L fermenter was not filled to capacity. A tight fitting lid was placed on the top of the 100 L fermenter.

These two reactions (10 kg and 50 kg) were allowed to proceed under ambient temperature conditions for approximately 72 hours. Another thermocouple was utilized to measure the temperature of the environment over the 72 hour period of fermentation. The temperature measurements are discussed in more detail in Example 11.

The 100 L fermenter used in for the 50 kg reaction had a cylindroconical shape and was equipped with a ½″ diameter drain located at the bottom for draining away any liquid that may be generated. The design of this 100 L fermenter was very similar to the type of silo which may be used for industrial application of this process. Seepage was one passive process which was utilized for product recovery where liquids drain from a mass of solid but porous material by gravity and collect at the bottom. A drain located in the proper location allows for easy collection of the liquid. For this experiment, the fermentation reaction was allowed to proceed for 72 hours and the drain at the bottom of the fermenter was closed. At approximately 72 hours the drain was opened and the liquids that had seeped to the bottom of the container were allowed to drain over a period of 1 hour. The volume of liquid collected from the drain was approximately 16 L.

The biomass slurry extract collected as seepage along with the liquefied plant biomass remaining in the fermenter was analyzed for ethanol and organic acid concentration using an HPLC with a BioRad HPX87-H column. Based on the large size of the reactions, a composite sample was prepared by mixing individual samples of approximately 500 g each taken from the top, middle and bottom portions of the reactor. This composite sample represented the approximate composition of the total reaction. The liquid seepage was purified with sedimentation and filtration prior to HPLC analysis. The solid samples collected from the 10 kg and 50 kg ferments were pressed and purified essentially as described in Example 3. For comparison purposes, two 100 g reactions are shown. The results of this analysis are shown in Table 16.

TABLE 16
Composition analysis of 50 kg fermentation.

| | | | Enzyme use | | | |
|---|---|---|---|---|---|---|
| | | Reaction | rate (ml | Lactic | Acetic | |
| | | mass | enzyme/100 g | Acid | Acid | Ethanol |
| Product Recovery Technique | Enzyme type | (kg) | biomass) | v/v % | v/v % | w/v % |
| Liquid Pressed From Plant Tissue | n/a | 0.1 | n/a | 0.4 | 0.6 | 3.6 |
| Liquid Pressed From Plant Tissue | n/a | 10 | n/a | 0.7 | 0.3 | 3.6 |
| Liquid Pressed From Plant Tissue | Celluclast | 0.1 | 2.6 | 2.9 | 0.5 | 4.4 |
| Liquid Pressed From Plant Tissue | Celluclast | 50 | 2.6 | 1.6 | 0.9 | 4.0 |
| Seepage | Celluclast | 50 | 2.6 | 2.5 | 0.4 | 4.3 |

The data show that the production of ethanol was enhanced through the application of cellulolytic enzymes presumably by the release of fermentable sugars from the cellulose containing biomass which are then converted to ethanol by inoculated yeast. The various sizes of reactions, from 100 g to 50 kg, produce very similar results for a particular experimental condition. The liquid seepage that was collected from the 50 kg reaction represents a very simple and energy efficient way to recover biomass slurry extract. The amount of ethanol and lactic acid remaining in the biomass slurry of the 50 kg reaction is reduced due to the fact that a large portion of these products have seeped from the reaction. The 16 L of extract collected represents approximately 30% of the starting mass of the reaction.

### Example 11

### Temperature

The production of liquefied plant biomass can be performed using standard silage storage tanks or other types of tanks which can hold plant biomass and liquid. Typically, these tanks are housed on farms and are located in exposed locations meaning that these tanks are not enclosed in barns or other types of buildings. To determine the effect of outdoor temperature on the inner temperature of plant biomass, temperature measurements were generated.

As outlined in Example 10, the ambient outdoor temperature was measured and compared to the temperature of the plant biomass inside the 5 gallon and 100 L containers. In addition, the performance of reactions exposed to ambient outdoor temperatures at 10 kg and 50 kg scale was measured and compared to the performance of 100 g reactions processed in a temperature controlled incubator. The ambient temperature outside during the 72 hour period ranged from approximately 15° C. to 30° C. Table 17 outlines the temperature readings recovered during the course of experimentation. There are many more temperature data points taken during this experiment and Table 17 is only a representative sample of measurements.

TABLE 17
Temperatures associated with Example 10 fermentations.

| | Reaction Temp | Reaction Temp | |
|---|---|---|---|
| | ° C. (50 kg | ° C. (10 kg | |
| | biomass + | biomass + | Ambient |
| Time/Date | enzyme) | no enzyme) | Temp ° C. |
| Sep. 16, 2010 13:38 | 26.7 | 31.6 | 27.7 |
| Sep. 16, 2010 16:38 | 33.4 | 33.2 | 23.6 |
| Sep. 16, 2010 19:38 | 35.5 | 32.9 | 25.8 |
| Sep. 16, 2010 22:38 | 35.7 | 31.8 | 22.9 |
| Sep. 17, 2010 1:38 | 35.6 | 30.5 | 20.9 |
| Sep. 17, 2010 4:38 | 35.3 | 28.8 | 17.5 |
| Sep. 17, 2010 7:38 | 34.6 | 27.2 | 18.2 |
| Sep. 17, 2010 10:38 | 34.1 | 26 | 21.1 |
| Sep. 17, 2010 13:38 | 33.9 | 26 | 26.7 |
| Sep. 17, 2010 16:38 | 33.1 | 26.2 | 28.9 |
| Sep. 17, 2010 19:38 | 32.9 | 27.4 | 29.3 |
| Sep. 17, 2010 22:38 | 31.4 | 27.4 | 22.4 |
| Sep. 18, 2010 1:38 | 30.8 | 27.2 | 18.9 |
| Sep. 18, 2010 4:38 | 30.3 | 26.3 | 16.9 |
| Sep. 18, 2010 7:38 | 29.7 | 24.8 | 14.3 |
| Sep. 18, 2010 10:38 | 29.5 | 23.6 | 17 |
| Sep. 18, 2010 13:38 | 29.8 | 23.4 | 23.7 |
| Sep. 18, 2010 16:38 | 29.1 | 23.5 | 27.4 |
| Sep. 18, 2010 19:38 | 29 | 24.5 | 27.4 |
| Sep. 18, 2010 22:38 | 27.9 | 24.9 | 21.9 |
| Sep. 19, 2010 1:38 | 27.3 | 25.1 | 20.1 |
| Sep. 19, 2010 4:38 | 27.2 | 24.5 | 18.1 |
| Sep. 19, 2010 7:38 | 27 | 23.7 | 17.5 |
| Sep. 19, 2010 10:38 | 26.8 | 23 | 18.6 |
| Sep. 19, 2010 13:38 | 27.2 | 22.9 | 23.2 |

This data demonstrates that the activity of yeast during the first 6 hours of the fermentation process generates heat which causes the plant biomass to warm to and achieve a temperature approximately 10° C. above ambient. The initial temperature can be influenced by the size of the reaction as the 50 kg reaction reached higher temperatures than the 10 kg reaction; however, it is expected that the reaction temperature would not exceed 39 degrees C. Over time, the fermentation reaction cools and the rate at which the reaction cools appears to be a function of the peak temperature achieved during fermentation and likely the size of the reaction as well. The larger the reaction the longer heat is retained within the biomass and the more insensitive the reaction is to changes in ambient temperatures. In addition, external temperature appears to influence the temperature of the plant biomass being introduced to the fermentation process and subsequently the initial temperature at the start of the fermentation. The temperature of the plant biomass will also have an impact on the speed of fermentation; colder temperatures will likely result in slower rates of production of end products such as ethanol and temperatures that are warmer will enhance reaction speed. It does not appear that cooler ambient temperatures affect the temperature of the process provided enough plant biomass is available. These data demonstrate that biomass slurry can be generated in ambient temperature using containers which are not temperature controlled provided a suitable reaction size is attained.

### Example 12

### Potential to Recover Biomass Slurry Extract (100 g Scale)

The reactions produced in Example 9 also served as test samples to study the potential to recover biomass slurry extract through the application of high pressure. At the end of the fermentation phase, the material in the fermentation vessels were introduced into a French Press and subjected to high pressure of approximately 500 psi essentially as described in Example 3. Table 18 outlines the percentage of liquid recovery, i.e. recovery of biomass slurry extract. The data is represented as a percentage of the liquid recovered at the end of the process where the liquid present in the plant biomass at the start of the process is set at 100%.

TABLE 18
Liquid recovery after French Press of fermentation reaction.

| | | Enzyme Dose | % recovery of |
|---|---|---|---|
| Reaction | | (ml enzyme/ | liquid fraction |
| Size | Enzyme Type | g biomass) | by press |
| 100 g | none | none | 75% |
| 100 g | Accellerase 1000 | 2.6 | 76% |
| 100 g | Accellerase 1000 | 5.2 | 81% |
| 100 g | Celluclast | 2.6 | 80% |
| 100 g | Celluclast | 5.2 | 80% |

This data demonstrates that the addition of any enzyme cocktail increases the recovery of liquid from the initial plant biomass and to some extent; this recovery can be influenced by the dose of the enzyme cocktail. In addition, this data demonstrates that approximately 75 to 80% of the liquid present in the biomass can be recovered by this method.

### Example 13

### Co-Product for Livestock Feed

Forage Sorghum, varieties FS350 and Graze n' Bale, were grown, harvested and processed essentially as described Examples 2 and 3. The plant biomass for this experiment was grown in the field and processed using the chipper shredder. Approximately 500 g of each variety of chopped forage sorghum plant biomass was combined with 20 g Ethanol Red dried yeast to form inoculated biomass. The biomass and yeast were well mixed by hand.

Enough inoculated biomass was partitioned to create 4 different 100 g reactions for each variety of forage sorghum. For each variety of forage sorghum the first reaction, which was considered the control, received an addition of 5.2 ml of distilled water. The remaining three reactions each received a unique combination of one or both of the two enzymes. The second 100 g reaction received an addition of 5.2 ml of Celluclast. The third reaction received an addition of 5.2 ml of Accellerase 1000. The fourth reaction received 2.6 ml additions of both Cellulcast and Accellerase 1000. The liquid additions were balanced for all reactions to facilitate comparison of the reaction products ethanol and organic acids. The above methods are essentially as described in Example 3.

The reactions were placed in 1 quart vacuum bags, sealed and allowed to ferment at 32° C. for approximately 72 hours. The compositions of reactions at the time of assembly in addition to the results of analysis of the liquid pressed from the reaction post fermentation are shown in Table 19.

TABLE 19
Composition analysis of fermented forage sorghum with enzyme added.

| | | | Celluclast | Accellerase | | | |
|---|---|---|---|---|---|---|---|
| | | | Enzyme added | Enzyme added | | | Total |
| | | | (ml enzyme/g | (ml enzyme/ | Crude | Acid Detergent | Digestible |
| Sorghum Variety | Processing Technique | Enzyme type | biomass) | g biomass) | Protein % | Fiber % | nutrients % |
| FS350 | fresh | none | 0 | 0 | 4.04 | 33.1 | 65 |
| FS350 | BX with yeast | none | 0 | 0 | 11.2 | 43.9 | 57.9 |
| FS350 | BX with yeast and enzyme | Celluclast | 5.2 | 0 | 13.5 | 33.2 | 65 |
| FS350 | BX with yeast and enzyme | Accellerase | 0 | 5.2 | 14 | 40.1 | 60.4 |
| FS350 | BX with yeast and enzyme | Celluclast + Accellerase | 2.6 | 2.6 | 13 | 38.6 | 61.4 |
| Graze n' Bale | fresh | none | 0 | 0 | 2.89 | 37.4 | 62.2 |
| Graze n' Bale | BX with yeast | none | 0 | 0 | 12.4 | 41.9 | 59.2 |
| Graze n' Bale | BX with yeast and enzyme | Celluclast | 5.2 | 0 | 10.7 | 37 | 62.5 |
| Graze n' Bale | BX with yeast and enzyme | Accellerase | 0 | 5.2 | 12.7 | 40.2 | 60.4 |
| Graze n' Bale | BX with yeast and enzyme | Celluclast + Accellerase | 2.6 | 2.6 | 13.4 | 37.3 | 62.3 |

The analysis demonstrates that this fermentation process produces a co-product that is suitable as a feed for livestock with or without the use of cellulolytic enzymes. The % protein in this co-product was greater than the fresh material. The feed produced as a co-product when cellulolytic enzymes were used showed a reduced concentration of Acid Detergent Fiber and increased concentration of Total Digestible Nutrients when compared to the co-product created without the use of such enzymes. It is possible that through the use of cellulolytic enzymes, the fiber content in the plant material was reduced facilitating an enhanced liquid product recovery as observed in Example 12.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method comprising;
a) collecting plant biomass, wherein the overall plant biomass comprises a moisture content between about 70% to about 95%,
b) chopping the plant biomass to produce a chopped plant biomass wherein the chopping comprises chopping the biomass to a theoretical cut length of less than 1.3 centimeters,
c) inoculating the chopped plant biomass with a ethanol-producing biocatalyst microbe to produce an inoculated chopped plant biomass;
d) placing the inoculated plant biomass into a chamber for at least 12 hours, wherein the inoculated plant biomass maintains a pH of greater than 3.5, and
e) providing a biomass slurry.

2. The method of claim 1, wherein the biomass slurry comprises ethanol

3. The method of claim 1, further comprising combining the inoculated plant biomass with at least one biomass-processing enzymes.

4. The method of claim 3, wherein the at least one biomass-processing enzyme is delivered by at least one of the methods selected from the group consisting of exogenous addition of the enzyme and transgenic expression of the enzyme in a plant or plant part.

5. The method of claim 1, wherein the addition of the ethanol-producing biocatalyst microbe reduces the amount of lactic acid produced in the liquefied plant biomass.

6. The method of claim 1, wherein the ethanol-producing biocatalyst microbe comprises a yeast.

7. The method of claim 6, wherein the yeast is a Saccharomyces cerevisiae.

8. The method of claim 1, wherein the biomass slurry comprises at least 2% ethanol.

9. The method of claim 1, wherein the biomass slurry comprises between about 2% to about 20% ethanol.

10. The method of claim 1, wherein the plant biomass material is derived from a one or more plants selected from the group consisting of maize, soybean, millet, milo, rye, wheat, triticale, oats, barley, rice, sorghum, sudangrass, switchgrass, Miscanthus, alfalfa, cotton, sisal, hemp, jute, turf grass, rape, sunflower, willow, eucalyptus, poplar, pine, willow, tobacco, clover, bamboo, flax, pea, radish, turnip, potato, sweet potato, cassaya, taro, beet, sugar beet, sugar cane, and canola.

11. The method of claim 1, wherein the inoculated plant biomass is placed in a chamber for a period of time from about 12 hours to about 21 days.

12. The method of claim 1, wherein the inoculated plant biomass is placed in a chamber for a period of time from about 12 hours to about 72 hours.

13. The method of claim 1, wherein the time from collecting the plant biomass to placing the inoculated plant biomass into a chamber is less than about 2 hours.

14. The method of claim 1, wherein heat is generated in the absence of any externally provided heat.
